(19) United States
(12) Patent Application Publication
(10) Pub. No.: US 2024/0216514 A1
(43) Pub. Date: Jul. 4, 2024

(54) NOVEL ANTITUMOR COMPOUNDS AND RELATED METHODS OF MANUFACTURE AND USE

(71) Applicant: KLOTHO THERAPEUTICS, INC., Las Vegas, NV (US)

(72) Inventors: Noriyuki KASAHARA, Belvedere Tiburon, CA (US); Michael Joseph Bishop, San Diego, CA (US); James Plante, Las Vegas, NV (US)

(21) Appl. No.: 18/554,617

(22) PCT Filed: Apr. 7, 2022
(86) PCT No.: PCT/US2022/071594

§ 371 Date: Oct. 9, 2023

Related U.S. Application Data
(60) Provisional application No. 63/171,747, filed on Apr. 7, 2021.

(51) Int. Cl. A61K 47/54

(52) U.S. Cl. CPC A61K 47/542

(57) ABSTRACT

Novel compounds and compositions including the same and methods of manufacturing and using the same, particularly for use as an anticancer/antitumor drug and/or for oncological indications. Novel compounds comprising or formed as conjugates of cisplatin, oxaliplatin, or other platinum moiety, particularly platinum(IV) moiety, and/with one or more moiety of 4-(phenylthio)butanoic acid (PTBA) or derivative of PTBA, and to compositions comprising the same, and to methods of manufacturing and using the same, particularly for use as an anticancer/antitumor drug and/or for oncological indications. Novel anticancer agents comprising a chemical conjugation of a platinum moiety and the therapeutically active ligand 4-(phenylthio)butyrate or a derivative thereof, as a drug or prodrug for cancer treatment.

### CROSS-REFERENCE TO RELATED APPLICATIONS

This application claims the benefit of and priority to U.S. Provisional Application No. 63/171,747, filed Apr. 7, 2021, titled Novel Antitumor Compounds and Related Methods of Manufacture and Use, the entirety of which is incorporated herein by specific reference.

### BACKGROUND

### 1. Technical Field

The present disclosure relates to novel compounds comprising or formed as conjugates of (1) a platinum atom and/with (2) 4-(phenylthio)butanoic acid (PTBA) or a derivative of PTBA, and to compositions comprising the same, and to methods of manufacturing and using the same, particularly for use as an anticancer/antitumor drug and/or for oncological indications. Specifically, the present disclosure relates to novel anticancer agents comprising a chemical conjugation of (1) a platinum atom (e.g., a platinum(IV) or platinum(II) moiety), or a platinum-based antineoplastic drugs (or “platin”), (2) one or more therapeutically-active ligand(s) of 4-(phenylthio)butyrate (or derivative thereof), and optionally, (3) one or more additional ligands, as a drug or prodrug for cancer treatment.

### 2. Related Technology

Platinum anticancer agents (or platins) are among the most widely used chemotherapeutic drugs and are administered in about 50% of all chemotherapeutic regimens. Cisplatin (cisdiamminedichloroplatinum [II]), in particular, has emerged as an effective chemotherapeutic in the treatment of testicular cancer, ovarian cancer, cervical cancer, breast cancer, bladder cancer, head and neck cancer, esophageal cancer, lung cancer, mesothelioma, brain tumors and neuroblastoma, and other cancers. Cisplatin is particularly effective against testicular cancer; its adoption has increased the cure rate from 10% to 85%. Other platins, such as oxaliplatin, carboplatin, etc. have also been effective in cancer treatment.

Cisplatin interferes with DNA replication, which kills the fastest proliferating cells, which in theory are cancerous. Specifically, cisplatin crosslinks DNA in several different ways, interfering with cell division by mitosis. The damaged DNA elicits DNA repair mechanisms, which in turn activate apoptosis when repair proves impossible. In 2008, for example, researchers were able to show that the apoptosis induced by cisplatin on human colon cancer cells depends on the mitochondrial serine-protease Omi/Htra2. Most notable among the changes in DNA are the 1,2-intrastrand cross-links with purine bases. These include 1,2-intrastrand d(GpG) adducts which form nearly 90% of the adducts and the less common 1,2-intrastrand d(ApG) adducts. 1,3-intrastrand d(GpXpG) adducts occur but are readily excised by the nucleotide excision repair (NER). Other adducts include inter-strand crosslinks and nonfunctional adducts that have been postulated to contribute to cisplatin's activity. Interaction with cellular proteins, particularly HMG domain proteins, has also been suggested as a mechanism of interfering with mitosis.

Cisplatin does, however, have a number of side effects that can limit its use. Nephrotoxicity (kidney damage), for example, is a major concern. Nephrotoxicity is a dose-limiting side effect; the dose of cisplatin may need to be reduced when the person's kidney function is impaired. Neurotoxicity (nerve damage) and other neurological side effects of cisplatin include visual perception and hearing disorder, which can occur soon after treatment begins. Nausea and vomiting is also common in cisplatin patients. Cisplatin is one of the most emetogenic chemotherapy agents. Ototoxicity (hearing loss) can occur in cisplatin patients. Cisplatin can also cause electrolyte disturbance, such as hypomagnesaemia, hypokalaemia and hypocalcaemia, and hemolytic anemia, which can be developed after several courses of cisplatin.

Accordingly, there are a number of short-comings in the art that can be addressed by the development, production, manufacture, and administration of new, platin-based chemotherapeutic drugs (“platins”).

### BRIEF SUMMARY

Embodiments of the present disclosure solve one or more of the foregoing or other problems in the art with novel conjugates of platinum (e.g., platinum(II) or platinum(IV)), and specifically novel conjugates of platinum and/with one or more ligands of 4-(phenylthio)butanoic acid (PTBA) or derivative(s) of 4-(phenylthio)butanoic acid (PTBA).

Some embodiments include a compound comprising (1) a platinum atom (e.g., a platinum(IV) or platinum(II) atom), (2) one or more ligands of 4-(phenylthio)butanoic acid (PTBA) or derivative(s) of 4-(phenylthio)butanoic acid (PTBA), and (3) one or more additional ligand(s) (e.g., other than 4-(phenylthio)butanoic acid (PTBA) or derivative(s) of 4-(phenylthio)butanoic acid (PTBA). Some embodiments include a compound comprising (1) a platinum-based anti-cancer or anti-tumor drug (e.g., cisplatin, oxaliplatin, etc.), (2) one or more ligands of 4-(phenylthio)butanoic acid (PTBA) or derivative(s) of 4-(phenylthio)butanoic acid (PTBA), and, optionally, (3) one or more additional ligand(s) (e.g., other than 4-(phenylthio)butanoic acid (PTBA) or derivative(s) of 4-(phenylthio)butanoic acid (PTBA). In some embodiments, the one or more additional ligand(s) can be or comprise OH, OCOCH₃

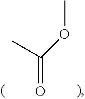

OCO(CH₂)₂CH₃,

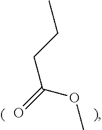

O(COCH((CH₂)₂CH₃)₂

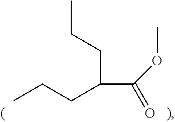

etc.

Embodiments of the present disclosure include chemical compounds (of the conjugate), compositions comprising the same, and methods of manufacturing and using the same, particularly for use as an anticancer/antitumor drug and/or for oncological indications. Specifically, embodiments of the present disclosure include novel anticancer agents comprising a chemical conjugation of a platinum atom (e.g., platinum (II) or (IV) moiety) or a chemical derivatives of an anticancer platinum drug (e.g., chemical derivatives of cisplatin or oxaliplatin, carboplatin, nedaplatin, phenanthriplatin, picoplatin, etc.), and the therapeutically active ligand 4-(phenylthio)butyrate (PTBA) (or derivative thereof), and optionally, one or more additional ligands, as a drug or prodrug for cancer treatment.

The present disclosure includes a family of novel complex (conjugate) molecules comprising (1) a platinum atom (e.g., platinum(II), platinum(IV), or an anticancer platinum drugs, such as cisplatin or oxaliplatin, carboplatin, nedaplatin, phenanthriplatin, picoplatin, etc.), combined, associated, and/or conjugated with (2) one or more ligands, such as the histone deacetylase 8 (HDAC8) inhibitor 4-(phenylthio)butyrate (PTBA), or derivative of 4-(phenylthio)butanoic acid (PTBA), and optionally, (3) one or more additional, chemically and/or sterically suitable ligands, as a drug or prodrug for cancer treatment. The present disclosure further includes pharmaceutical compositions comprising such molecules or compounds. The present disclosure further includes use of the molecules or compounds, or the pharmaceutical compositions, for the treatment of a variety of cancers or tumors. The presence of more than one active moiety and the ability to permit simultaneous release, in cancer cells, of the active moieties, each acting via a different anticancer pathway permits eradication of cancer cells by acting on, or activating, more than one different cellular targets, increasing the chances of effective cancer cells eradication, including those resistant to anticancer drugs, and patient survivability. Thus, the present disclosure provides platinum based multifunctional compounds, each containing a platinum center and one or more moiety of 4-(phenylthio)butyrate.

In at least one aspect, the present disclosure provides a compound comprising at least one platinum atom associated to one or more 4-(phenylthio)butyrate ligand,. In at least one aspect, the present disclosure provides a compound comprising a platinum(IV) atom bound, conjugated, and/or associated to one or more 4-(phenylthio)butyrate ligand.

In various embodiments, the compound of the present disclosure is in the form of a prodrug, capable of releasing the 4-(phenylthio)butyrate (PTBA) ligand as a therapeutically active molecule, thereby affecting anticancer activity. In various embodiments, the compound of the present disclosure is in the form of a prodrug, capable of releasing a platinum or platinum-based anti-cancer or anti-tumor compound as a therapeutically active molecule, thereby affecting anticancer activity.

In another aspect, there is provided a Pt-anticancer agent comprising one or more 4-(phenylthio)butyrate (PTBA) moieties. In some embodiments, the Pt-anticancer agent comprises at least two 4-(phenylthio)butyrate ligands; in other embodiments, the Pt-anticancer agent comprises at least three 4-(phenylthio)butyrate ligands, and yet in other embodiments, the Pt-anticancer agent comprises at least four 4-(phenylthio)butyrate ligands.

In some embodiments, the Pt-anticancer agent is associated to 1 or 2 or 3 or 5 or 6 (separate) ligands of 4-(phenylthio)butyrate. Non limiting, illustrative embodiments of exemplary compounds of the present disclosure are depicted in FIG. 1.

In some aspects or embodiments of the present disclosure, the 4-(phenylthio)butyrate ligand associated with the Pt atom is a monodentate ligand. In some aspects or embodiments of the present disclosure, each of the ligands associated with the Pt atom is a monodentate ligand. Where the complex of the present disclosure includes a single 4-(phenylthio)butyrate ligand, the complex may comprise in addition one or more polydentate ligand or one or more additional monodentate ligand, being different from 4-(phenylthio)butyrate. The “polydentate ligand”, being a ‘donor group’, is a ligand having more than one atom that can associate (or link, coordinate) directly to the Pt atom in a complex according to the present disclosure; wherein a “monodentate” ligand forms a single bond with the metal atom. In some embodiments, the complex has at least one monodentate ligand. In some embodiments, the complex has at least one polydentate ligand. In some embodiments, the at least one polydentate ligand is a bidentate ligand. In some embodiments, the at least one polydentate ligand is a tridentate ligand. In some embodiments, the at least one polydentate ligand is a tetradentate ligand.

In some embodiments, the complex of the present disclosure comprises two monodentate 4-(phenylthio)butyrate ligands. The complex of the present disclosure may be in any structural isomerization or stereoisomerization or optical isomers. In some embodiments, the complex is a cis isomer. In some embodiments, the complex is a trans isomer. In some embodiments, the complex is a mer-isomer. In some embodiments, the complex is afac-isomer. In some embodiments, the complex is in an octahedral geometry, wherein at least two of the ligands are in the axial positions of the octahedral complex. In some embodiments, the complex is in an octahedral geometry, wherein two ligands are 4-(phenylthio)butyrate ligands positioned in the axial positions of the octahedral complex. In some embodiments, the complex is in an octahedral geometry, wherein one of the ligands positioned in an axial position of the octahedral complex is 4-(phenylthio)butyrate.

Various embodiments of the present disclosure can be represented by Formula A, below:

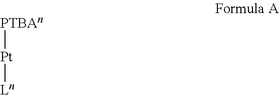

As illustrated in Formula A, embodiments of the present disclosure can comprise a platinum atom (“Pt”), one or more ligands or moieties of 4-(phenylthio)butyrate, or a derivative of 4-(phenylthio)butanoic acid (“PTBA”), and one or more (optional) additional moieties or ligand (“L”).

In some embodiments, compounds of the present disclosure may be according to one of Formulas B-G, below, where “Pt” represents a platinum atom, “PTBA” represents the histone deacetylase 8 (HDAC8) inhibitor, 4-(phenylthio)butyrate (PTBA), or derivative of 4-(phenylthio)butanoic acid (PTBA), and “L” represents a ligand:

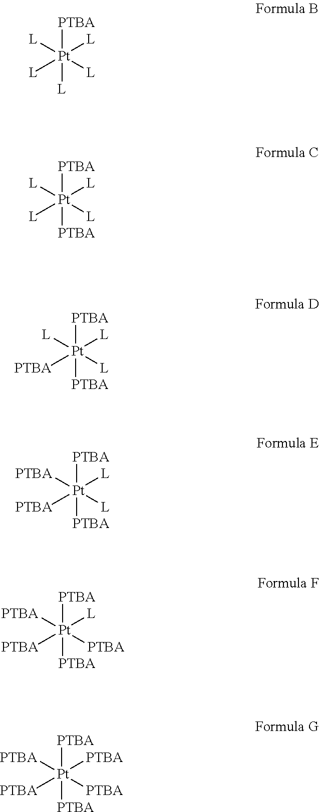

Those skilled in the art will appreciate that, while Formulas B-G, above each illustrate six (6) ligands—represented as various combinations of “PTBA” and “L”, that other combinations are also contemplated herein. In particular, various embodiments of the present disclosure include four (4) ligands or moieties associated with the platinum atom, “Pt”, which can be any suitable combination of “PTBA” and “L”.

In some embodiments, “PTBA” in Formulas A-G, above, can be or comprise:

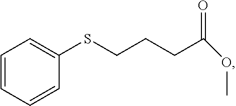

a derivative of

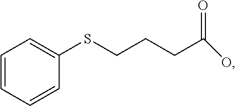

etc. Derivatives of can comprise 1-5 substituents at the phenyl ring. Such substituents at the phenyl ring can be or comprise, for example, halo (Cl, F, Br, etc.), alkyl (methyl, ethyl, propyl, isopropyl, etc.), alkoxy (e.g., methoxy), hydroxy (e.g., OH), hydroxyalkyl (e.g. hydroxymethyl (CH₂OH)), CF3, or CN. Derivatives of can also or alternatively comprise one or more substitutions in the backbone (e.g., at a backbone methylene). Such backbone substituents can be or comprise, for example, alkyl (methyl, ethyl, propyl, isopropyl, etc.), alkoxyl (e.g., methoxy, ethoxy, etc.), or halo (e.g., fluoro, chloro, bromo, etc.).

In some embodiments, Pt (with ligands “L”, as necessary) in Formulas A-G (particularly, Formula C), above, can form, or be, or comprise (substituted) cisplatin, oxaliplatin, carboplatin, nedaplatin, phenanthriplatin, picoplatin, or any other suitable platinum-based chemotherapeutic or other drug.

In some embodiments, “L” in Formulas B-G, above, can be nothing or null.

In some embodiments, the (additional) ligand (“L”) in Formulas A-G, above, can be or comprise: Cl, NH₂, OH, OCOCH₃

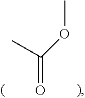

OCO(CH₂)₂CH₃,

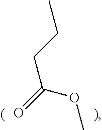

O(COCH((CH₂)₂CH₃)₂

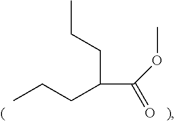

etc. In some embodiments, two adjacent ligands (“L”) in Formulas A-G, above, can form:

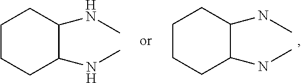

etc.

A first aspect or embodiment of the present disclosure includes a compound according to Formula I:

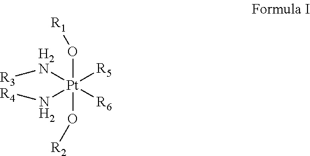

wherein:
R₁ is 4-(phenylthio)butyryl

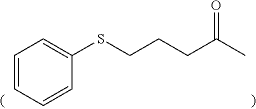

or a derivative of 4-(phenylthio)butyryl;
R₂ is selected from the group consisting of 4-(phenylthio)butyryl, H, COCH₃

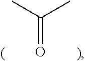

CO(CH₂)₂CH₃,

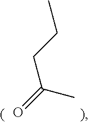

and (COCH((CH₂)₂CH₃)₂

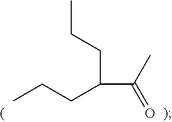

R₃ is H or, together with R₄, forms 1,2-cyclohexyl

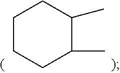

R₄ is H or, together with R₃, forms 1,2-cyclohexyl

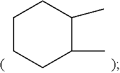

R₅ is Cl or, together with R₆, forms and

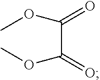

R₆ is Cl or, together with R₅, forms

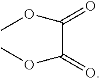

Additional aspect or embodiments of the present disclosure include the following:

2. The compound of aspect or embodiment 1, wherein R₃ is H, R₄ is H, R₅ is Cl, and R₆ is Cl.
3. The compound of aspect or embodiment 2, wherein R₂ is 4-(phenylthio)butyryl or H.
4. The compound of aspect or embodiment 2, wherein R₂ is 4-(phenylthio)butyryl.
5. The compound of aspect or embodiment 2, wherein R₂ is H.
6. The compound of aspect or embodiment 2, wherein R₂ is selected from the group consisting of COCH₃

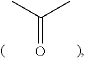

CO(CH₂)₂CH₃,

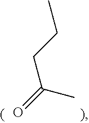

and (COCH((CH₂)₂CH₃)₂

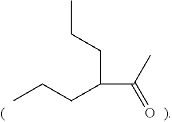

7. The compound of aspect or embodiment 2, wherein R₂ is COCH₃

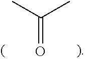

8. The compound of aspect or embodiment 2, wherein R₂ is CO(CH₂)₂CH₃,

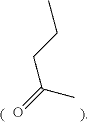

9. The compound of aspect or embodiment 2, wherein R₂ is (COCH((CH₂)₂CH₃)₂

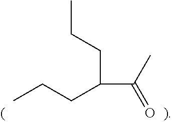

10. The compound of aspect or embodiment 1, wherein R₂ is 4-(phenylthio)butyryl or H.
11. The compound of aspect or embodiment 1, wherein R₂ is 4-(phenylthio)butyryl.
12. The compound of aspect or embodiment 1, wherein R₂ is H.
13. The compound of aspect or embodiment 1, wherein R₂ is selected from the group consisting of COCH₃

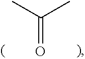

CO(CH₂)₂CH₃,

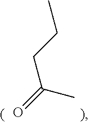

and (COCH((CH₂)₂CH₃)₂

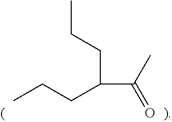

14. The compound of aspect or embodiment 1, wherein R₂ is COCH₃

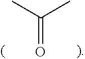

15. The compound of aspect or embodiment 1, wherein R₂ is CO(CH₂)₂CH₃,

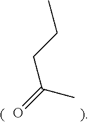

16. The compound of aspect or embodiment 1, wherein R₂ is (COCH((CH₂)₂CH₃)₂

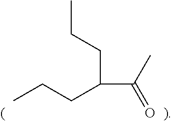

17. The compound of aspect or embodiment 1, wherein R₃ is H and R₄ is H.
18. The compound of aspect or embodiment 1, wherein R₅ is Cl and R₆ is Cl.
19. The compound of aspect or embodiment 1, wherein:
R₃, together with R₄, forms 1,2-cyclohexyl

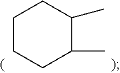

R₄, together with R₃, forms 1,2-cyclohexyl

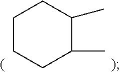

R₅, together with R₆, forms

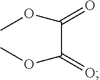

and
R₆, together with R₅, forms

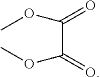

20. The compound of aspect or embodiment 19, wherein R₂ is H or COCH₃

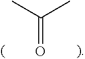

21. The compound of aspect or embodiment 19, wherein R₂ is H.
22. The compound of aspect or embodiment 19, wherein R₂ is COCH₃

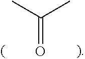

23. The compound of aspect or embodiment 1, wherein:
R₃, together with R₄, forms 1,2-cyclohexyl

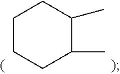

and
R₄, together with R₃, forms 1,2-cyclohexyl

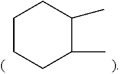

24. The compound of aspect or embodiment 23, wherein R₂ is H.
25. The compound of aspect or embodiment 23, wherein R₂ is COCH₃

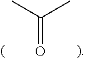

26. The compound of aspect or embodiment 1, wherein:
R₅, together with R₆, forms

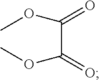

and
R₆, together with R₅, forms

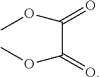

27. The compound of aspect or embodiment 26, wherein R₂ is H.
28. The compound of aspect or embodiment 26, wherein R₂ is COCH₃

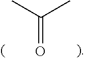

29. The compound of any one of aspect or embodiments 1-28, wherein R₁ is 4-(phenylthio)butyryl.
30. The compound of any one of aspect or embodiments 1-28, wherein R₁ is a derivative of 4-(phenylthio)butyryl, wherein the derivative comprises 4-(phenylthio)butyryl having one or more phenyl ring substituents, optionally selected from the group consisting of halo (Cl, F, Br, etc.), alkyl (methyl, ethyl, propyl, isopropyl, etc.), alkoxy (e.g., methoxy), hydroxy (e.g., OH), hydroxyalkyl (e.g. hydroxymethyl (CH₂OH)), CF₃, and CN.
31. The compound of any one of aspect or embodiments 1-28, wherein R₁ is a derivative of 4-(phenylthio)butyryl, wherein the derivative comprises 4-(phenylthio)butyryl having one or more backbone substituents or backbone methylene substitutes, optionally selected from the group consisting of alkyl (methyl, ethyl, propyl, isopropyl, etc.), alkoxyl (e.g., methoxy, ethoxy, etc.), or halo (e.g., fluoro, chloro, bromo, etc.).
32. The compound of aspect or embodiment 1, wherein the compound is selected from the group consisting of:

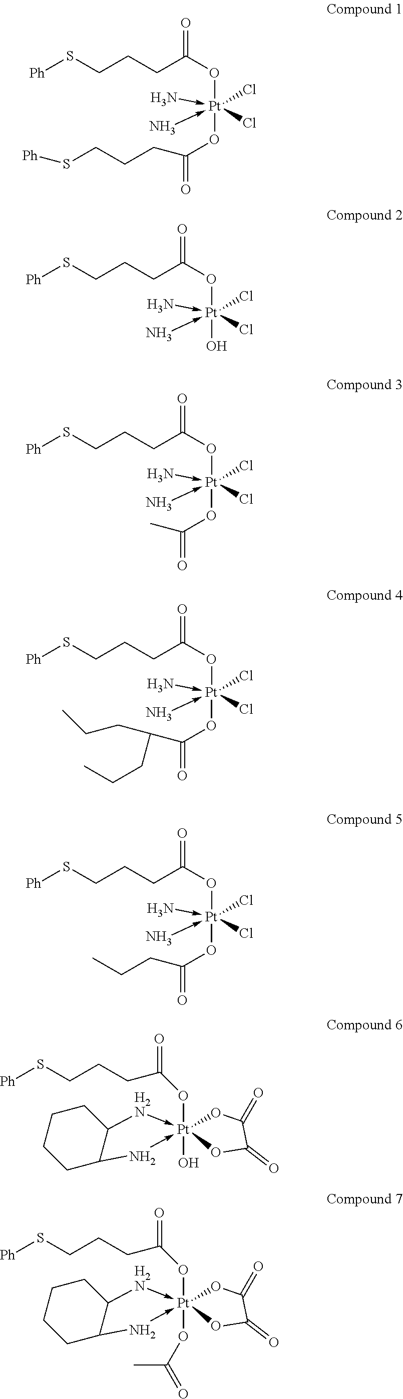

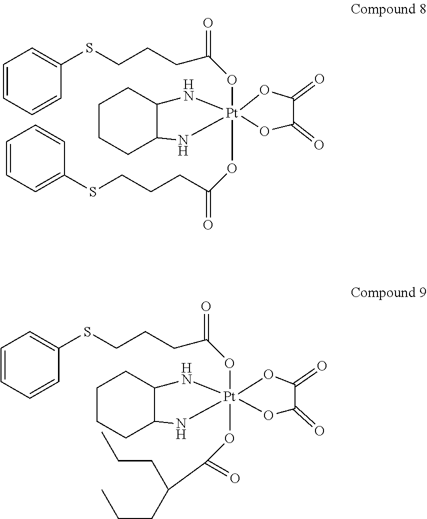

33. A compound, comprising:
a platinum-based chemotherapeutic molecule, preferably selected from the group consisting of cisplatin and oxaliplatin; and
one or more molecule or moiety of 4-(phenylthio)butanoic acid (PTBA) conjugated to the platinum-based chemotherapeutic molecule; and
optionally, one or more additional ligand conjugated to the platinum-based chemotherapeutic molecule.
   34. A compound, comprising:
a platinum-based chemotherapeutic molecule, preferably selected from the group consisting of cisplatin and oxaliplatin; and
a molecule or moiety of 4-(phenylthio)butanoic acid (PTBA) conjugated to the platinum-based chemotherapeutic molecule; and
a ligand conjugated to the platinum-based chemotherapeutic molecule.
   35. The compound of aspect or embodiment 34, wherein the ligand is selected from the group consisting of PTBA, OH, OCOCH₃

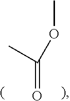

OCO(CH₂)₂CH₃,

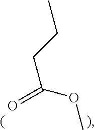

and O(COCH((CH₂)₂CH₃)₂

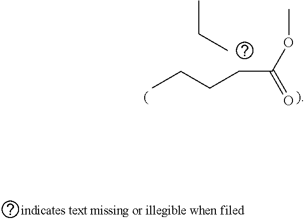

36. A composition comprising: the compound of any one of aspect or embodiments 1-35; and a carrier or excipient.
37. The composition of aspect or embodiment 36 for use as a medicament for the treatment of cancer or a tumor.
38. The composition of aspect or embodiment 36 for use as a medicament for the treatment of cancer or a tumor, wherein the cancer or tumor is suitable for or susceptible to treatment with cisplatin or oxaliplatin.
39. The composition of aspect or embodiment 36 for use in the treatment of cancer or a tumor.
40. The composition of aspect or embodiment 36 for use in the treatment of cancer or a tumor, wherein the cancer or tumor is suitable for or susceptible to treatment with cisplatin or oxaliplatin.
41. A method, comprising administering the composition of aspect or embodiment 36 to a subject.
42. A method for treating cancer or a tumor, the method comprising administering the composition of aspect or embodiment 36 to a subject having the cancer or tumor.
43. The method of aspect or embodiment 42, wherein the cancer or tumor is suitable for or susceptible to treatment with cisplatin or oxaliplatin.
44. A method of inhibiting histone deacetylase (HDAC) or HDAC activity, the method comprising administering the composition of aspect or embodiment 36 to a subject.
45. A method of reducing tumor size, the method comprising administering the composition of aspect or embodiment 36 to a subject having a tumor.
46. The method of aspect or embodiment 45, wherein the tumor is suitable for or susceptible to treatment with cisplatin or oxaliplatin.
47. A method, comprising administering the compound of any one of aspect or embodiments 1-35 to a subject.
48. A method for treating cancer or a tumor, the method comprising administering the compound of any one of aspect or embodiments 1-35 to a subject having the cancer or tumor.
49. The method of aspect or embodiment 48, wherein the cancer or tumor is suitable for or susceptible to treatment with cisplatin or oxaliplatin.
50. A method of inhibiting histone deacetylase (HDAC) or HDAC activity, the method comprising administering the compound of any one of aspect or embodiments 1-35 to a subject.
51. A method of reducing tumor size, the method comprising administering the compound of any one of aspect or embodiments 1-35 to a subject having a tumor.
52. The method of aspect or embodiment 51, wherein the tumor is suitable for or susceptible to treatment with cisplatin or oxaliplatin.
53. The compound of any one of aspect or embodiments 1-35 for use in the manufacture of a medicament for the treatment of cancer or tumor.
54. The compound of any one of aspect or embodiments 1-35 for use in the manufacture of a medicament for the treatment of cancer or a tumor, wherein the cancer or tumor is suitable for or susceptible to treatment with cisplatin or oxaliplatin.
55. The compound of any one of aspect or embodiments 1-35 for use in the treatment of cancer or a tumor.
56. The compound of any one of aspect or embodiments 1-35 for use in the treatment of cancer or a tumor, wherein the cancer or tumor is suitable for or susceptible to treatment with cisplatin or oxaliplatin.

Non-limiting, illustrative embodiments of the present disclosure include exemplary Compounds 1-9, below:

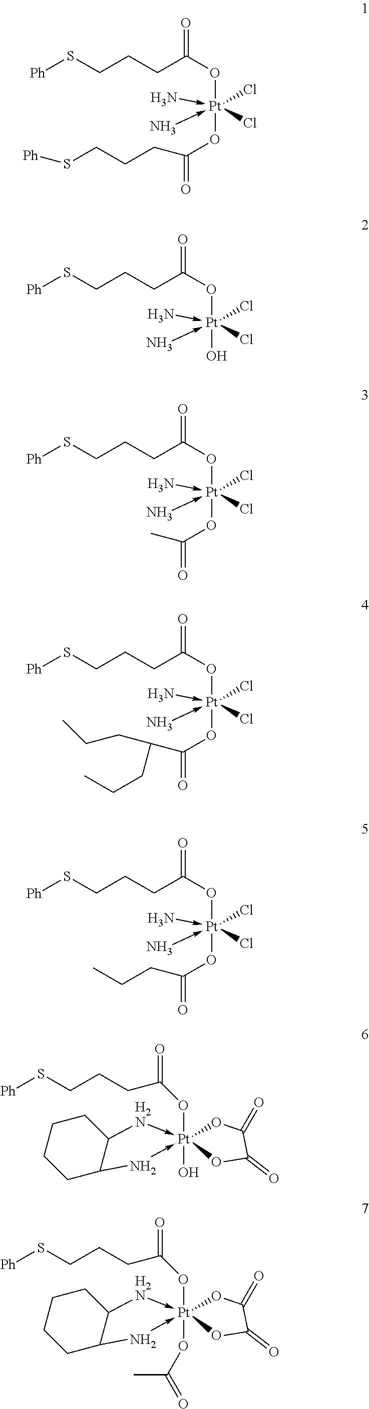

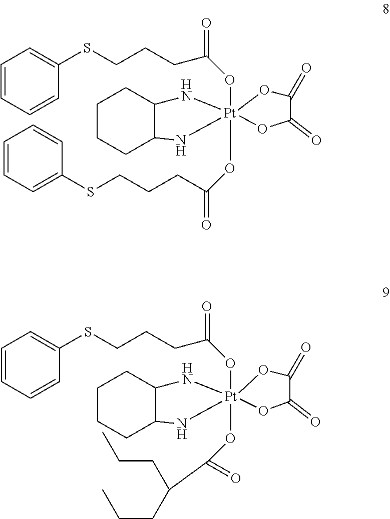

Those skilled in the art will appreciate that various conventions exist for illustrating bonds, associations, conjugations, and so forth. In various embodiments of the present disclosure, for example, such bonds, associations, conjugations, etc. may be illustrated using a line, an arrow, a solid arrowhead, a dashed arrowhead, etc. Those skilled in the art will understand the chemical components associated with each marking and, where necessary, apply a more correct marking, as appropriate.

Some embodiments may include any of the features, options, and/or possibilities set out elsewhere in the present disclosure, including in other aspects or embodiments of the present disclosure. It is also noted that each of the foregoing, following, and/or other features described herein represent a distinct embodiment of the present disclosure. Moreover, combinations of any two or more of such features represent distinct embodiments of the present disclosure. Such features or embodiments can also be combined in any suitable combination and/or order without departing from the scope of this disclosure. Thus, each of the features described herein can be combinable with any one or more other features described herein in any suitable combination and/or order. Accordingly, the present disclosure is not limited to the specific combinations of exemplary embodiments described in detail herein.

Embodiments of the present disclosure are designed to be effective for use as a chemotherapeutic, antitumor compound, or anticancer compound, or a combination (1) chemotherapeutic, antitumor compound, or anticancer compound, and (2) histone deacetylase 8 (HDAC8) inhibitor, or inhibitor of histone deacetylase 8 (HDAC8) activity. Inhibition of HDAC8 activity may have several downstream and/or therapeutic effects. Those skilled in the art will appreciate that cancers and other disease(s) or condition(s) that is/are caused, worsened, or exacerbated, in whole or in part, by (or associated with) high or excessive HDAC8 activity, may be addressed and/or treated (post-diagnosis or prophylactically) by administration of the novel compound(s), or composition(s) comprising the same, disclosed herein. Other HDACs and HDAC inhibitors are also contemplated herein.

Those skilled in the art will further appreciate that combinatorial (multi-drug) chemotherapy treatment regimens are also contemplated herein. Illustratively, embodiments of the present disclosure can include combinatorial treatment regimens (or methods) comprising treatment with (or administering) one or more of the inventive compounds (platinum-PTBA conjugates) of the present disclosure, with another chemotherapeutic or other drug known to be combinable with or co-administered with platinum-based chemotherapeutics. For example, cisplatin is known to be co-administered with drugs, such as gemcitabine, fluorouracil or 5-fluorouracil, etoposide, nimotuzumab, paclitaxel, vinorelbine, etc. Similarly, Oxaliplatin is known to be co-administered with drugs, such as, for example, capecitabine, 5-fu/leucovorin (folfox), xeloda, panitumumab, trastuzumab, pemetrexed, etc. Radiation therapy is also common co-treatment with cisplatin. Accordingly, one or more of the foregoing can be co-administered or co-treated with the inventive compounds of the present disclosure.

Additional features and advantages of exemplary embodiments of the present disclosure will be set forth in the description that follows, and in part will be obvious from the description, or may be learned by the practice of such exemplary embodiments. The features and advantages of such embodiments may be realized and obtained by means of the instruments and combinations particularly pointed out in the appended claims. These and other features will become more fully apparent from the following description and appended claims, or may be learned by the practice of such exemplary embodiments as set forth hereinafter.

### BRIEF DESCRIPTION OF THE DRAWINGS

In order to describe the manner in which the above-recited and other advantages and features of the present disclosure can be obtained, a more particular description of the embodiments briefly described above will be rendered by reference to specific embodiments thereof which are illustrated in the appended drawings. For better understanding, the like elements have been designated by like reference numbers throughout the figure(s). Understanding that these drawings depict only typical embodiments of the disclosure and are not therefore to be considered to be limiting of its scope, the disclosure will be described and explained with additional specificity and detail through the use of the accompanying drawing(s) in which:

FIG. 1 depicts nine (9) illustrative compounds according to the present disclosure.

FIG. 2 illustrates the antitumor activity of an exemplary treatment of the present disclosure.

FIGS. 3A-3C illustrate mean IC50s (uM) of inventive compounds of the present disclosure in various cancer cell lines.

FIG. 4A-4C illustrate dose response curve of selected cell lines comparing cisplatin to inventive conjugate compounds of the present disclosure.

### DETAILED DESCRIPTION

Before describing various embodiments of the present disclosure in detail, it is to be understood that this disclosure is not limited only to the specific parameters, verbiage, and description of the particularly exemplified systems, methods, and/or products that may vary from one embodiment to the next. Thus, while certain embodiments of the present disclosure will be described in detail, with reference to specific features (e.g., configurations, parameters, properties, steps, components, ingredients, members, elements, parts, and/or portions, etc.), the descriptions are illustrative and are not to be construed as limiting the scope of the present disclosure and/or the claimed invention. In addition, the terminology used herein is for the purpose of describing the embodiments, and is not necessarily intended to limit the scope of the present disclosure and/or the claimed invention.

Unless defined otherwise, all technical and scientific terms used herein have the same meaning as commonly understood by one of ordinary skill in the art to which the present disclosure pertains.

Various aspects of the present disclosure, including systems, methods, and/or products may be illustrated with reference to one or more embodiments, which are exemplary in nature. As used herein, the terms “embodiment” mean “serving as an example, instance, or illustration,” and should not necessarily be construed as preferred or advantageous over other aspects disclosed herein. In addition, reference to an “ embodiment” of the present disclosure or invention is intended to provide an illustrative example without limiting the scope of the invention, which is indicated by the appended claims.

As used in this specification and the appended claims, the singular forms “a,” “an” and “the” each contemplate, include, and specifically disclose both the singular and plural referents, unless the context clearly dictates otherwise. For example, reference to a “protein” contemplates and specifically discloses one, as well as a plurality of (e.g., two or more, three or more, etc.) proteins. Similarly, use of a plural referent does not necessarily require a plurality of such referents, but contemplates, includes, specifically discloses, and/or provides support for a single, as well as a plurality of such referents, unless the context clearly dictates otherwise.

As used throughout this disclosure, the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

The term “condition” refers to any disorder, disease, injury, or illness, as understood by those skilled in the art, that is manifested or anticipated in a patient. Manifestation of such a condition can be an early, middle, or late stage manifestation, as known in the art, including pre-condition symptoms, signs, or markers. Anticipation of such a condition can be or include the predicted, expected, envisioned, presumed, supposed, and/or speculated occurrence of the same, whether founded in scientific or medical evidence, risk assessment, or mere apprehension or trepidation.

The term “patient,” as used herein, is synonymous with the term “subject” and generally refers to any animal under the care of a medical professional, as that term is defined herein, with particular reference to (i) humans (under the care of a doctor, nurse, or medical assistant or volunteer) and (ii) non-human animals, such as non-human mammals (under the care of a veterinarian or other veterinary professional, assistant, or volunteer).

For the sake of brevity, the present disclosure may recite a list or range of numerical values. It will be appreciated, however, that where such a list or range of numerical values (e.g., greater than, less than, up to, at least, and/or about a certain value, and/or between two recited values) is disclosed or recited, any specific value or range of values falling within the disclosed values or list or range of values is likewise specifically disclosed and contemplated herein.

To facilitate understanding, like references (i.e., like naming of components and/or elements) have been used, where possible, to designate like elements common to different embodiments of the present disclosure. Similarly, like components, or components with like functions, will be provided with similar reference designations, where possible. Specific language will be used herein to describe the exemplary embodiments. Nevertheless it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

While the detailed description is separated into sections, the section headers and contents within each section are for organizational purposes only and are not intended to be self-contained descriptions and embodiments or to limit the scope of the description or the claims. Rather, the contents of each section within the detailed description are intended to be read and understood as a collective whole, where elements of one section may pertain to and/or inform other sections. Accordingly, embodiments specifically disclosed within one section may also relate to and/or serve as additional and/or alternative embodiments in another section having the same and/or similar products, methods, and/or terminology.

Provided herein are compounds useful as a chemotherapeutic, antitumor compound, or anticancer compound, or as a combination (1) chemotherapeutic, antitumor compound, or anticancer compound, and (2) histone deacetylase (HDAC) inhibitor or inhibitor of HDAC8 activity, preferably HDAC8 inhibitor, or inhibitor of histone deacetylase 8 (HDAC8) activity. Efficacy of the compounds is demonstrated below.

Two of the major drawbacks involved in administration of chemotherapeutics are (1) the need for intravenous administration, which requires hospitalization, incurring significant costs, and (2) the ability of tumors to develop resistance to these drugs. One approach clinicians use for the treatment of unresponsive cancer patients is to use drug cocktails that act by different mechanisms.

Histone deacetylase (HDAC) inhibitors are emerging as a new class of anticancer drugs that can alter gene transcription and exert antitumor effects such as growth arrest, differentiation, apoptosis, and inhibition of tumor angiogenesis. HDACs are dysregulated in many cancers, making them a therapeutic target for the treatment of cancer. Histone deacetylase inhibitors (HDACi), a novel class of small-molecular therapeutics, are now approved by the Food and Drug Administration as anticancer agents. While they have shown great promise, resistance to HDACi is often observed and furthermore, HDACi have shown limited success in treating solid tumors. However, the combination of HDACi with standard chemotherapeutic drugs has demonstrated promising anticancer effects in both preclinical and clinical studies.

Histone deacetylase (HDAC) inhibitors remove acetate from acetylated ϵ-amino groups of lysines in histones and other proteins. Removal of acetyl groups from histones causes global condensation of chromatin and suppression of gene expression, and accordingly, HDACs play an important role in epigenetic regulation. HDACs are also termed lysine deacetylases (KDACs, from lysine's amino acid letter designation “K”), since these enzymes also remove acetyl groups from lysines of numerous nuclear and cytosolic proteins, affecting both gene transcription and cellular signaling. Disturbances in HDAC normal expression or activity result in aberrant cellular functions, and are associated with various diseases. Therefore, HDACs are emerging as important molecular targets for therapeutic intervention, including cancer. Four classes of HDACs are known, classes I-IV that constitute 18 different isotypes which are dependent on either zinc (classical HDACs: I, II and IV) or nicotinamide adenine dinucleotide (NAD, Sirtuins: III), respectively, as cofactors. The Class I/II HDAC inhibitor Vorinostat (suberoylanilide hydroxamic acid, or SAHA) was the first HDAC inhibitor to be approved for clinical use by the FDA for treatment of refractory cutaneous T-cell lymphoma [9]. However, Vorinostat broadly inhibits most Class I and Class I HDAC isotypes. In fact, all the FDA-approved HDAC inhibitors are broad-spectrum HDAC inhibitors, hitting more than one isotype. This contributes to the significant adverse side effects, and restricts the utility and efficacy of these compounds for use as anti-cancer drugs.

Because of the associated adverse side effects of pan-HDAC inhibitors, recently much effort has been devoted to developing inhibitors of specific HDAC isotypes. HDAC8, a Class I isotype, is gaining importance due to its prominent role in specific cancer subtypes as T-cell lymphoma, childhood neuroblastoma and other diseases, such as X-linked intellectual disability and parasitic infections. HDAC8 is a Zn++-dependent class I HDAC, identified as a 42 kDa protein containing 377 amino acids (aa). Found both in nucleus and cytoplasm, many of the cellular deacetylation targets of HDAC8 identified so far are known to localize into the nucleus (e.g., AT-rich interactive domain-containing protein 1A, estrogen receptor alpha, hEST1B and structural maintenance of chromosome 3 [SMC3]). It is an X-linked protein in humans and had diverged early from other class I members implying significant functional specialization. Indeed, the independence of cofactors for activity and the absence of the protein binding domain reflect specific characteristics of HDAC8, which are not present in other members of this class. Some active site features such as the highly flexible L1 loop, the conserved aspartate 101 and regulation by serine 39 phosphorylation add to the distinct functional specialization of HDAC8.

Although histones are viewed as the classical HDAC substrates, there is ample proof for non-histone substrates as well. For HDAC8, there is conflicting evidence concerning histones as in vivo deacetylation substrates. While there is data that hyperacetylation results as a consequence of HDAC8 inhibitor treatment or overexpression leads to low acetylation , sophisticated mass spectroscopy analyses have not always detected histones as HDAC8 substrates. There is a possibility that HDAC8 might prefer deacetylation of only very particular sites, which could be masked by global acetylation patterns or likely be dependent on the cell type. However, in vitro, deacetylation of histone variants like H2A/H2B, H3 and H4 by HDAC8 has been observed. Even short peptides derived from histones are excellent in vitro substrates, and studies using deacetylation sites in such peptides has contributed to the understanding of sequence specificity for HDAC8 deacetylation sites: for example, deacetylation occurs at lysines 14, 16, 20 on histone H4-derived peptides and K(Ac)RHR is a preferred motif in this histone subtype.

Besides histones, a large number of non-histone candidates have been recognized as substrates or interaction partners of HDAC8. SMC3, ERRα and p53 have been shown by RNAi or pharmacological inhibition of HDAC8 to be direct cellular substrates with clear concentration-dependent hyperacetylation effects. Mass spectrometric analysis of cellular lysates after treatment with HDAC8 inhibitors has established several other novel proteins, such as retinoic acid-induced 1, zinc-finger Ran binding domain-containing protein 2, AT-rich interactive domain-containing protein 1A, nuclear receptor coactivator 3, thyroid hormone receptor-associated protein 3, peroxiredoxin 6, phosphoglycerate mutase 1, high mobility group protein B1, and Parkinson protein 7, as HDAC8 deacetylation targets.

Peptide arrays have revealed an even broader range of candidate non-histone protein substrates, indicating further physiological substrates to be discovered. Some non-histone proteins, such as inv(16) fusion protein, CREB, DEC1, Hsp20, human ever-shorter telomeres 1B (hEST1B) and α-actin have been found to be associated with HDAC8. However, it is unclear whether these proteins are direct acetylation targets or form a part of complex in which HDAC8 acts as a scaffold. For instance, HDAC8 coimmunoprecipitated with both CREB and protein phosphatase 1; ectopic expression of HDAC8 decreased CREB activity. Similarly, HDAC8 colocalized and immunoprecipitated with smooth muscle myosin heavy chain and possibly connects to inv(16) fusion protein via this domain. In this case, pharmacological inhibition with trichostatin A affected transcriptional activities of the inv(16) fusion protein. Both for CREB and inv(16), such evidence suggests HDAC8 can interact with these proteins as deacetylation substrate or as members of a cooperative complex.

More clues for scaffolding function come from interactions with a-actin. Colocalization of HDAC8 with a-actin and subsequent knockdown effects in human smooth muscle cells (including phenotypic changes such as smaller cells with loss of contractibility or spreading) without changes in global a-actin acetylation points toward a scaffolding

behavior of HDAC8 rather than deacetylase activity in those cases [49]. Furthermore, DNA-ChIP analysis demonstrated a colocalization of HDAC8 with the transcription factor DEC1. Modulation of HDAC8 expression affected DEC1- and DEC1-regulated TAp73 transcription factor implying a recruiting role of DEC-1 with HDAC8. Most probably, deacetylation and scaffolding functions for HDAC8 are intertwined. Moreover, non-epigenetic roles of HDAC8 are also possible. Recent studies demonstrating an ability of HDAC8 to control Notch stability or binding to miRNA miR-216b point to an ever-increasing complexity of the role of HDAC8 in cells.

Mutations in the HDAC8 active site lead to loss of activity and are for example, linked with Cornelia de Lange syndrome (CdLS). Another fundamental epigenetic role of HDAC8 is the control of skull morphogenesis; it has been shown that deletion of HDAC8 leads to perinatal lethality in mice due to skull instability. Other novel biological roles of HDAC8 are still being discovered in bone differentiation, repression of interleukin β, and toxin-induced resistance of macrophages. HDAC8 has been implicated in viral infections, and HDAC8 inhibition reduced infection load in parasitic disease models. In the field of oncology, HDAC8 is known to be dysregulated in T-cell lymphoma, childhood neuroblastoma and gastric cancer.

Specifically, HDAC8 is expressed in colon, breast, lung, hepatocellular carcinoma, gastric cancer, pancreas tumor tissue, metastatic melanoma, acute myeloid leukemia (AML), acute lymphocytic leukemia (ALL) as well as in childhood tumors of the nervous system, such as neuroblastoma. Although HDAC8 is also expressed in corresponding normal tissue, it displays a significant upregulation in aggressive stage 4 neuroblastomas. In invasive breast tumor cells, HDAC8 is among the three HDAC family members that are upregulated and driving invasiveness. Conversely, RNAi-mediated knockdown of HDAC8 expression in human lung, colon, leukemia, gastric adenocarcinoma and cervical cancer cell lines inhibits tumor cell proliferation. Upregulation of HDAC8 promotes proliferation and inhibits apoptosis in hepatocellular carcinoma and gastric cancer.

HDAC8 expression itself can be regulated by the lipogenic transcription factor sterol regulatory element binding protein-1 and the SOX-family of transcription factors. SREBP-1 links lipid metabolism, insulin resistance and cancer development and has been shown to directly upregulate HDAC8 in models of nonalcoholic fatty liver disease-associated hepatocellular carcinoma. Microarray analyses of adult T-cell leukemia/lymphoma revealed that HDAC8 expression in these tumor cells is regulated by SOX4, which directly activates the HDAC8 promoter. SOX4 is a transcription factor, which is required for B-lymphocyte development, and also during development of the sympathetic nervous system. So far, several mechanisms of action are described for HDAC8. In colon cancer, for example, the Bcl-2-modifying factor (BMF) has been identified to be a direct target gene of HDAC8 repression and HDAC8 derecruitment is sufficient to activate the target gene. Furthermore, STAT3 associates and cooperates with HDAC8 to repress BMF transcription. BMF has an important function in the execution of apoptosis triggered by the HDAC inhibitory metabolite methylselenopyruvate, which is a competitive inhibitor of HDAC8.

In addition, HDAC8 has been implicated in contributing to tumorigenesis by regulating telomerase activity. In AML, the inv(16) fusion protein associates with HDAC8 in order to repress the transcription of AMLI-regulated genes. Several studies describe a link between HDAC8 and the tumor suppressor p53. The interaction of inv(16) with HDAC8, for example, causes HDAC8-mediated deacetylation and inactivation of p53 in AML leukemia stem cells which promotes tumor cell transformation. Knockdown of HDAC8 expression elevates the expression and acetylation of p53 in hepatocellular carcinoma cells, resulting in decreased cell proliferation and activation of apoptosis. Besides the regulation of p53, HDAC8 is also involved in the regulation of another transcription factor of the p53 family, p73, which also plays a key role in many biological processes, such as neuronal development and tumorigenesis.

In 2006, the FDA approved the first HDAC inhibitor—suberoylanilide hydroxamic acid (SAHA, Vorinostat), a broad-spectrum HDAC inhibitor, to treat the rare, refractory cutaneous T-cell lymphoma (CTCL). Vorinostat (SAHA) is a weaker inhibitor of HDAC8 (micromolar range) than of HDACs 1-3 (nanomolar range). Trichostatin A, on the other hand, completely inhibits HDAC8 activity only at 5 μM, though in Molt-4 acute lymphoblastic leukemia cells it attenuates HDAC8 expression. Besides the known clinical side effects of SAHA (e.g. leukopenia, thrombopenia, fatigue, diarrhea) which limit its usefulness as an anti-cancer drug, this inhibitor has very recently been reported to promote epithelial mesenchymal transition and cell motility in triple negative breast cancer cell culture. This negative side effect was reported to be dependent on HDAC8.

In terms of adverse side effects, the targeting of a single enzyme would be superior to broad spectrum HDAC inhibition when applied in an appropriate tumor type that displays oncogenic dependency on that particular HDAC family member. The specific involvement of HDAC8 in cancers such as leukemia and childhood neuroblastoma indicates significant therapeutic potential. Especially in neuroblastoma and T-cell lymphoma, both entities with a clear correlation of HDAC8 activity with the disease, specific targeting of HDAC8 may represent a highly efficacious treatment with limited off-target effects. For example, in neuroblastoma, HDAC8 expression correlates with aggressive Stage 4 tumors, and accordingly is associated with poor outcomes. Selective HDAC8 inhibition in neuroblastoma cell lines induces signs of differentiation, such as the outgrowth of neurofilament-positive

neurite-like structures [58]. In this context it should be noted that targeting of other HDAC family members in this tumor entity affects completely different processes (e.g., apoptosis or autophagy). Hence, selective inhibitors of HDAC8 may be a potential new class of drugs for differentiation therapy of cancer, while avoid the adverse side effects incurred by broad-spectrum HDAC inhibitors.

In clinical trials, HDAC inhibitors have been utilized in combination with platinum anticancer drugs. An ongoing trial (phase 2) entitled “Valproic Acid and Platinum based Chemoradiation in Locally Advanced Head and Neck Squamous Cell Carcinoma” aimed to evaluate if the addition of valproic acid to standard platinum based chemo-radiation as definitive treatment of locally advanced head and neck squamous cell carcinoma could improve treatment outcomes, such as response rate. A Phase I Clinical Trial of Vorinostat in combination with Gemcitabine plus Platinum in patients with advanced Non-Small Cell Lung Cancer was completed in 2011.

Another approach to treating unresponsive cancer patients is to improve the therapeutic profiles of the anticancer drug. For instance, a complex of Pt(IV) prodrug with valproic acid, a specific histone deacetylase inhibitor, and its experiments in treatment of human carcinoma cell lines has been proposed and demonstrated.

### Chemical Synthesis

All chemicals, reagents and solvents were obtained from commercial vendors, such as Enamine, Sigma-Aldrich, and Fisher Scientific.

Cisplatin and oxaliplatin are commercially available and can be easily synthesized in the lab. 4-(phenylthio)butyric acid is also commercially available.

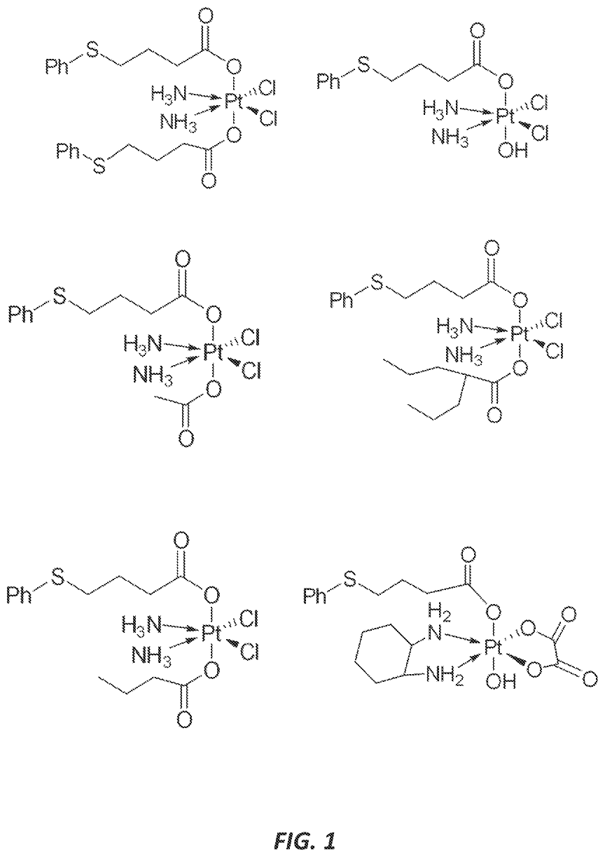

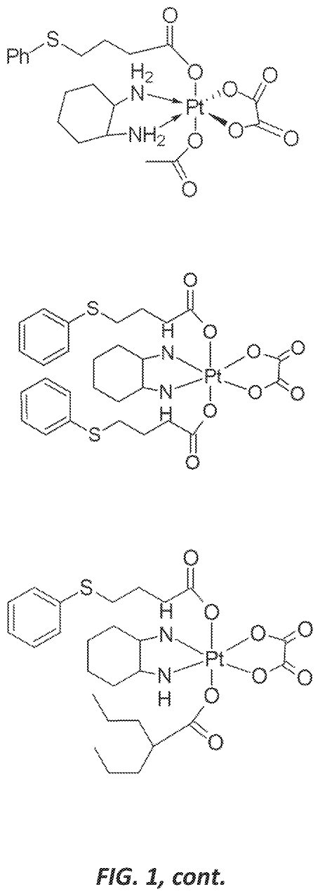

Non-limiting examples of illustrative compounds according to embodiments of the present disclosure are depicted in FIG. 1.

Generally, the synthesis of (fully symmetric) compounds is done by standard procedures, as described herein and understood to those skilled in the art. In brief, cisplatin or oxaliplatin are oxidized with H₂O₂ and are then reacted with an excess of an anhydride yield the desired compounds.

The compounds with two different groups on the oxygens of the oxidized platin are prepared by oxidizing cisplatin (or similar Pt complex) in acetic acid (or another carboxylic acid) to yield [Pt(NH₃)₂(OH)(OAc)Cl₂] (or similar appropriate Pt complex, and/or where OAc is replaced with the appropriate carboxylate), or by reacting the oxidized cisplatin (or similar Pt complex) with an amount of an anhydride to give one carboxylate on the Pt and leaving a free —OH on the Pt. This compound with the free —OH can be a compound of the invention, or can be further reacted with 1.5 equivalents of an anhydride DMF or acetonitrile, or other suitable solvent, to yield ctc-[Pt(NH₃)₂(L)(L₂)Cl₂] (or similar appropriate Pt complex), where L and L₂ are different carboxylate ligands.

Cisplatin and oxaliplatin are oxidized with H₂O₂ to yield the Pt(IV) complexes, as shown in Scheme 1:

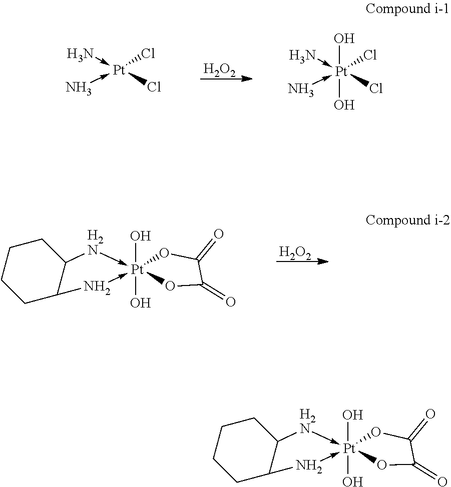

### Synthesis of Intermediate (compound i1): cis,cis,trans-[Pt(NH₃)₂Cl₂(OH)₂]

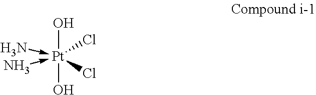

To stirred mixture of cisplatin (0.100 g, 0.33 mmol), accurately weighed in a 5 ml round bottom flask, and distilled water (0.2 mL), was slowly added dropwise H₂O₂ (30% w/v, 1.0 mL) at 70° C. for 5 hours. After completion of the reaction, the reaction solution was cooled to room temperature and then placed at 4° C. overnight, then the supernatant was removed by centrifugation to give a pale-yellow solid. The solid was washed twice with distilled water, ethanol, and ether, and then dried in vacuo to afford compound 4 as a pale-yellow solid (0.075 g, 68% yield).

### Synthesis of Intermediate: 4-(phenylthio)butanoic anhydride

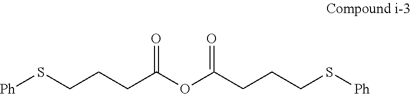 [SMILES: O=C(CCCSc1ccccc1)OC(=O)CCCSc1ccccc1]

4-(Phenylthio)butanoic acid (1) (1.7 g, 4.52 mmol) and N,N-dicyclohexyl carbodiimide (DCC, 0.472 g, 2.26 mmol) were dissolved in chloroform (15 mL) and stirred overnight at room temperature. The dicyclohexylurea by-product was removed by Celite filtration. The crude mixture was dissolved in DCM (30 mL), concentrated under reduced pressure, and filtered, and the procedure was repeated until no urea was observed to obtain 1.4 g of 4-(phenylthio)butanoic anhydride (84% yield).

### Example 1. Ctc-[Pt(NH₃)₂(4-phenylthiobutyrate)2Cl₂]

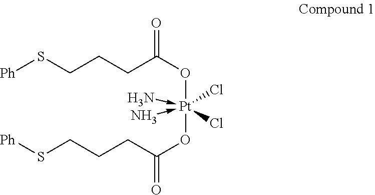

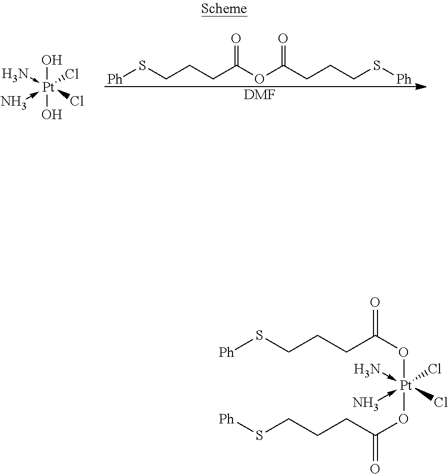

cis, cis, trans-[Pt(NH₃)₂Cl₂(OH)₂] (100 mg, 0.3 mmol) was suspended in N,N-dimethylformamide (1 mL) and 4-phenylthio butyric anhydride was added (287.5 mg, 0.75 mmol). The reaction mixture was stirred for 1 h (complete dissolution) at 40° C. The solvent was evaporated at reduced pressure. The residue was re-dissolved in acetone and diethyl ether was added to precipitate the compound. The precipitate was collected by filtration, washed twice with diethyl ether and dried in vacuo to obtain crude desired product. The combined batches from two separate 100 mg reaction runs were purified by HPLC (2-10 min 20-60% acetonitrile 30 mL/min ((loading pump 4 mL acetonitrile) column: SunFire C18 100×19 5 microM) to give 83 mg (95% purity). ¹H NMR (400 MHZ, dmso) δ 7.36-7.26; (m, 8H), 7.21-7.12; (m, 2H), 6.88-6.31; (m, 6H), 3.00; (t, J=7.4, 7.4 Hz, 4H), 2.37; (t, J=7.1, 7.1 Hz, 4H), 1.75; (m, 4H). MS: 689.0 [M+H]⁺.

### Example 2: Ctc-[Pt(NH₃)₂(OH)(4-phenylthiobutyrate)Cl₂]

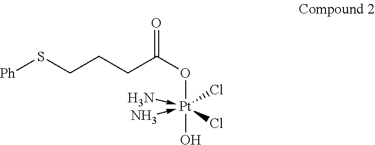

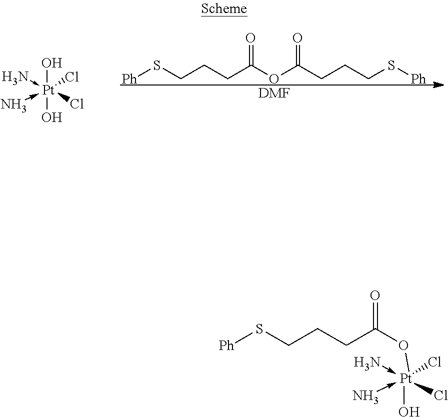

cis, cis, trans-[Pt(NH₃)₂Cl₂(OH)₂] (0.075 g, 0.22 mmol) and 4-(phenylthio)butyric anhydride (0.092 g, 0.24 mmol) were stirred in DMSO (7 mL) for 1 day at RT. The resulting mixture was filtered, the mother liquor was treated with ether affording a two-phase system. The ether phase was removed after centrifugation, and the product was isolated by HPLC to give Ctc-[Pt(NH₃)₂(OH)(4-phenylthiobutyrate)Cl₂] as a pale-yellow solid (0.0555 mg, 47.5% yield). ¹H NMR (600 MHZ, dmso) δ 7.35-7.25; (m, 4H), 7.14; (t, J=6.7, 1H), 6.09-5.75; (m, 6H), 2.98; (t, J=7.5 Hz, 2H), 2.29; (t, J=7.1, 2H), 1.73; (m, 2H). MS (ESI): mass calcd. for C₁₀H₁₆Cl₂N₂O₃PtS 510, m/z found, 511.0 [M+H]+.

### Example 3: Ctc-[Pt(NH₃)₂(C₂H₃0₂)(4-phenylthiobutyrate)Cl₂]

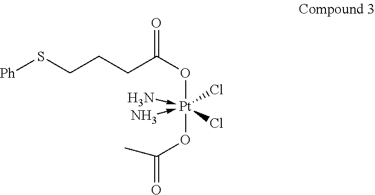

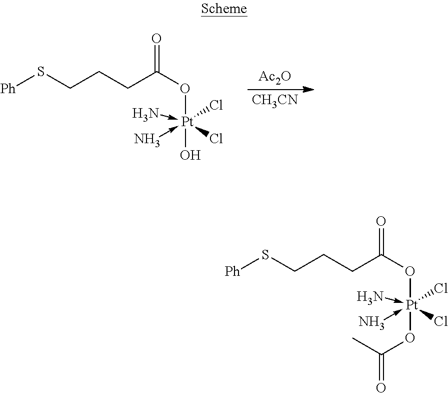

Compound 2 (0.100 g, 0.196 mmol) was suspended in acetonitrile (3 mL) and acetic anhydride (2 mL) was added to the suspension. After 2 h at RT, the resulting mixture was turned to a bright yellow solution, filtered, concentrated under reduced pressure and precipitated with a mixture of diethyl ether and petroleum ether (1:1). The solid (90 mg, 65% pure) was purified by HPLC (2-10 min 10-100% acetonitrile 30 mL/min ((loading pump 4 mL acetonitrile) column: SunFire C18 100×19 5 microM) and then lyophilized to give 0.018 g (97.5% purity by LCMS) of compound Ctc-[Pt(NH₃)₂(C₂H₃0₂)(4-phenylthiobutyrate)Cl₂] (32% yield). ¹H NMR (400 MHZ, dmso) δ 7.34-7.30; (m, 4H), 7.20-7.14; (m, 1H), 6.71-6.33; (m, 6H), 2.99; (t, J=7.4, 7.4 Hz, 2H), 2.37; (t, J=7.1, 7.1 Hz, 2H), 1.91; (s, 3H), 1.74; (p, J=7.7, 7.7, 7.4, 7.4 Hz, 2H). MS: 553.0 [M+H]⁺.

### Example 4: Ctc-[Pt(NH₃)₂(valproate)(4-phenylthiobutyrate)Cl₂]

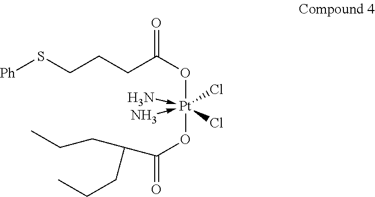

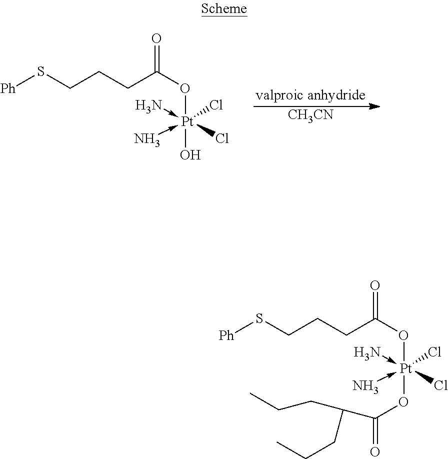

Compound 2 (0.200 g, 0.392 mmol) was suspended in acetonitrile (6 mL) and valproic anhydride (0.2113 g, 0.78 mmol) was added to the suspension. After 4 h at RT, the resulting mixture was turned to a bright yellow solution, filtered, concentrated under reduced pressure, affording a yellow sticky solid which was precipitated with diethyl ether. The solid was separated by centrifugation, and purified by HPLC (2-10 min 40-100% acetonitrile 30 mL/min ((loading pump 4 mL acetonitrile) column: SunFire C18 100×19 5 microM) to obtain 51mg of Ctc-[Pt(NH₃)₂(valproate)(4-phenylthiobutyrate)Cl₂] (yield 20.4%). ¹H NMR (400 MHZ, cd₃od) δ 7.36; (d, J=7.5 Hz, 2H), 7.28; (t, J=7.7, 7.7 Hz, 2H), 7.15; (t, J=7.3, 7.3 Hz, 1H), 3.01; (t, J=7.3, 7.3 Hz, 2H), 2.53; (t, J=7.1, 7.1 Hz, 2H), 2.45-2.39; (m, 1H), 1.89; (p, J=7.2, 7.2, 7.2, 7.2 Hz, 2H), 1.65-1.42; (m, 2H), 1.42-1.27; (m, 6H), 0.91 (t, J=7.0, 7.0 Hz, 6H). MS: 637.2 [M+H]⁺.

### Example 5: Ctc-[Pt(NH₃)₂(butyrate)(4-phenylthiobutyrate)Cl₂]

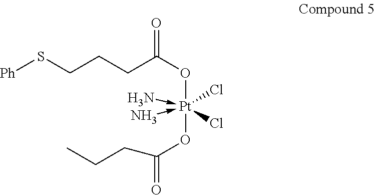

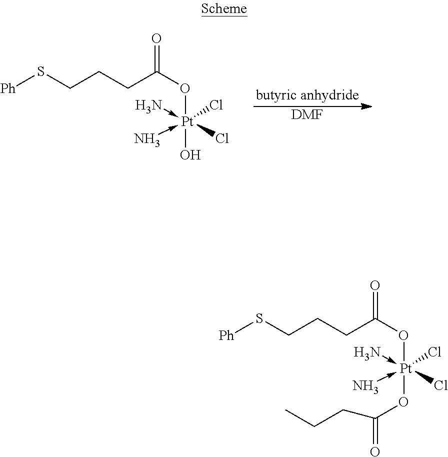

Compound 2 (0.100 g, 0.196 mmol) was suspended in DMF (1 mL) and butyric anhydride (1 mL) was added to the suspension. After 2 h at 60° C., the resulting mixture turned to a bright yellow solution, was filtered, concentrated under reduced pressure and precipitated with a mixture of diethyl ether and petroleum ether (1:1). The residue was dissolved in acetone and precipitated with ether, collected and dried to obtain 13 mg of Ctc-[Pt(NH₃)₂(butyrate)(4-phenylthiobutyrate)Cl₂] (11% yield). ¹H NMR (400 MHZ, DMSO) δ 7.36-7.26; (m, 4H), 7.21-7.09; (m, 1H), 6.75-6.29; (m, 6H), 2.99; (t, J=7.4, 7.4 Hz, 2H), 2.36; (t, J=7.1, 7.1 Hz, 2H), 2.19; (t, J=7.3, 7.3 Hz, 2H), 1.74 (m, 2H), 1.46; (m, 2H), 0.86; (m J=7.3, 7.3 Hz, 3H). MS: 580.0 [M+H]⁺.

### Example 6: Synthesis of Ctc-[Pt((1R,2R)-1,2-cyclohexanediamine-N,N′) (OH)(4-phenylthiobutyrate) (oxalato(2-)-O,O′)]

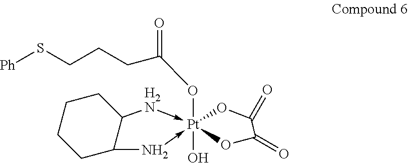

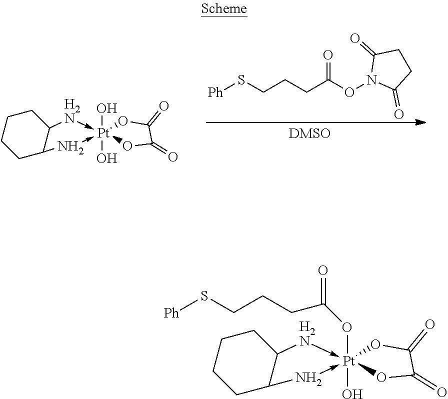

Oxaliplatin (200 mg, 0.5 mmol) was suspended in water (6 mL) in a 25 mL one neck round bottom flask equipped with magnetic stirring bar, and 33% hydrogen peroxide (7.1 mL, 75 mmol, 150 eq) was added to the suspension. The reaction mixture was stirred for 1 h at 50° C. The solution was evaporated on a rotary evaporator. The product was precipitated by adding ethanol (2 mL) and the formed precipitate filtered. The solid was washed with ethanol (2 mL) and diethyl ether (2×5 mL) and dried under vacuum (1 mbar) to give 181.5 mg (84%) of product. In a one neck 50 mL round bottom flask equipped with a magnetic stirrer to a cooled (with cold water) solution of phenythiobutyric acid (3.93 g, 20 mmol, 1 eq) and N-hydroxysuccinimide (2.53 g, 22 mmol, 1.1 eq) in DMF (25 mL) was added in portions EDCI·HCl (4.22 g, 22 mmol, 1.1 eq). The reaction was stirred for 36 h, then diluted with water (250 mL, HPLC) and extracted with EtOAc (3×25 mL). The combined extracts were washed with 5% citric acid (50 mL) and 3% NaHCO3 (50 mL), dried over Na2So4 (25 g) and evaporated The crude product (4.72 g, 80%) was used further as is. Ctc-[Pt((1R,2R)-1,2-cyclohexanediamine-N,N′) (OH)₂(oxalato(2-)-O,O′)] (0.1 g, 0.23 mmol, 1 eq) was added to the solution of phenylthiobutyric acid NHS ester (68 mg, 0.23 mmol, 1 eq) in DMSO (10 mL, pre-dried over mol sieves overnight) in a 25 mL one neck round bottom flask equipped with stirring magnet under N₂ atmosphere. The mixture stirred for at rt for 3 days and additional 20 mg of Pt-complex and the mixture stirred overnight. The DMSO was evaporated, the residue (0.28 g) purified by column chromatography on reverse phase silica gel (C18, h=10 cm, d=3.5 cm, elution with MeCN/H2O=40:60), to give 28 mg (20%) of product. MS: 610.2 [M+H]⁺.

### Example 7: Synthesis of Ctc-[Pt((1R,2R)-1,2-cyclohexanediamine-N,N′) (C₂H₃0₂)(4-phenylthiobutyrate) (oxalato(2-)-O,O′)]

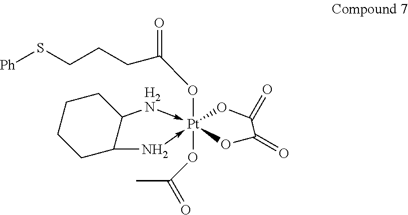

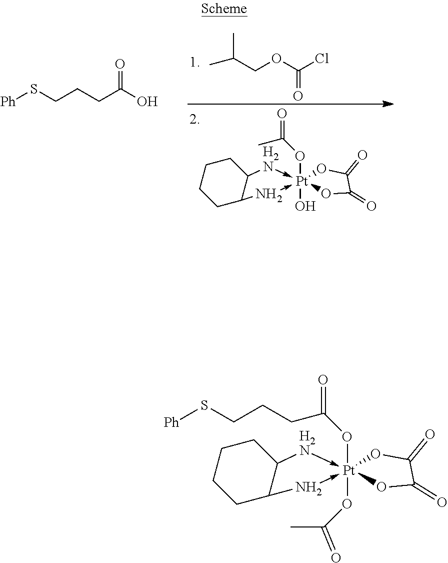

Preparation of Ctc-[Pt((1R,2R)-1,2-cyclohexanediamine-N,N′) (C₂H₃0₂)(OH) (oxalato(2-)-O,O′)]: Oxaliplatin (200 mg, 0.50 mmol) was suspended in glacial acetic acid (10 mL) in a 25 mL one neck round bottom flask equipped with magnetic stirring bar, and 33% hydrogen peroxide (140 μL, 1.5 mmol) was added to the suspension. The reaction mixture was stirred in the dark at ambient temperature and monitored by HPLC/MS After 18 h (overnight), the acetic acid was removed from the reaction mix under vacuum and the product was washed with ethyl acetate (2×5 mL) and collected as a white solid (260 mg, >100%, mixture of mono and diacetate). Isobutyl chloroformate (61 μL, 0.465 mmol, 1.1 eq) was added with stirring to an ice-water cooled solution of phenylthiobutanoic acid (VAS-23, 91.2 mg, 0.465 mmol, 1.1 eq) and Et3N (129 uL, 0.930 mmol, 2.2 eq) in DMF (4 mL) in a 10 mL one neck round bottom flask equipped with magnetic stirring bar. After 30 min, Ctc-[Pt((1R,2R)-1,2-cyclohexanediamine-N,N′) (C₂H₃0₂)(OH) (oxalato(2-)-O,O′)] (200 mg, 0.105 mmol, 1 eq) was added as solid. The reaction mixture was allowed to warm to rt and stirred at rt overnight. Reaction was checked by HPLC/MS . When completed, evaporated to give 380 mg of crude product. The crude product was stirred 5 times with Et2O (5-10 mL), organic phase was discharged each time. The residual material (270 mg) was purified by preparative TLC (SiO2, 1 mm thick layer, 20×20 cm, solvent system DCM/MeOH 9:1) to give 80 mg (29%) of Ctc-[Pt((1R,2R)-1,2-cyclohexanediamine-N,N′) (C₂H₃0₂)(4-phenylthiobutyrate) (oxalato(2-)-O,O′)]. MS: 652.3 [M+H]⁺.

### Example 8. Tumor Cell Growth Inhibition by Select Compounds of the Invention

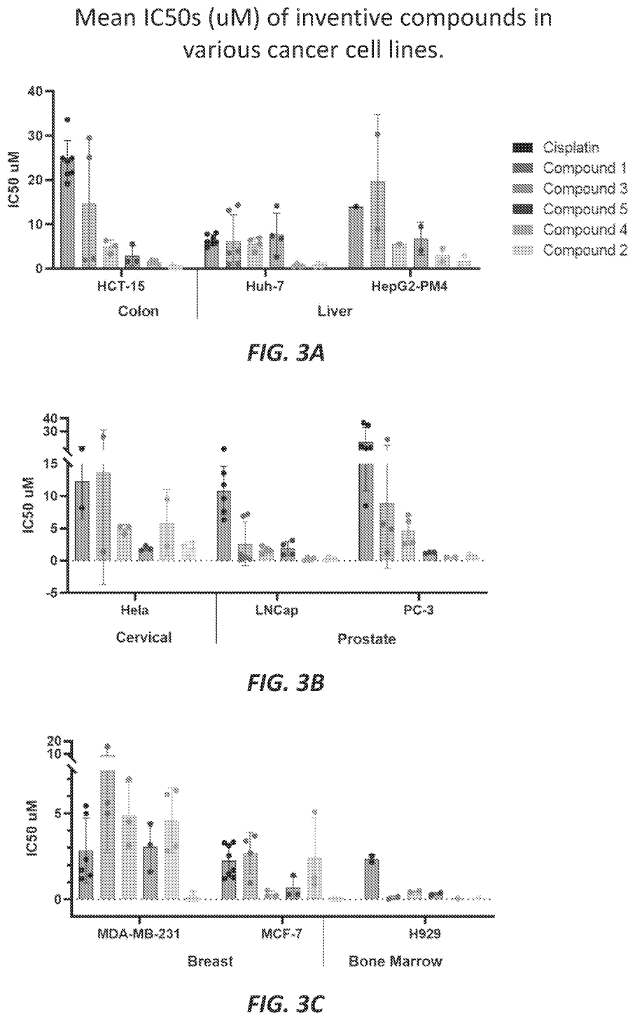

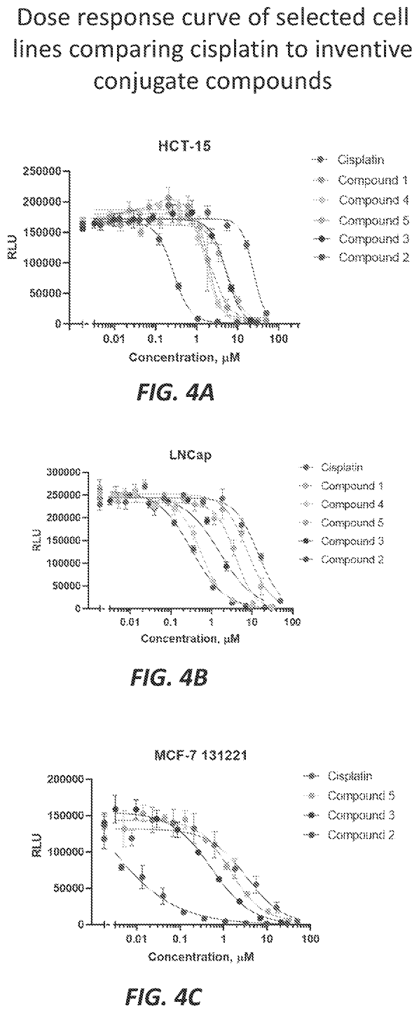

To support the potential of compounds of the invention as antitumor agents, their ability to inhibit tumor cell growth across a range of cancer types was evaluated, compared to cisplatin (See FIGS. 3A-3C and FIGS. 4A-4C). Cancer cells were seeded at optimized density on 384-well plates in appropriate media for 24 hours in triplicate for each test article. Titrated compounds or cisplatin were added to cultures for 72 hours with 3-fold dilutions totaling 9 concentrations starting from 30 uM for compounds or 50 uM for cisplatin (see FIGS. 3A-3C). Cell viability was then determined by CellTiter-Glo® Luminescent Cell Viability Assay from Promega, Madison Wisconsin. A dose response curve (see FIGS. 4A-4C) was fitted from luminance activity and the IC₅₀ for each test article was calculated using a four-parameter variable slope in GraphPad Prism version 8.0.0 for Windows, GraphPad Software, San Diego, California USA, www.graphpad.com. Conjugates were more cytotoxic to cancer cells compared to cisplatin alone, some were particularly noteworthy in colon cancer (HCT-15), prostate cancer (PC-3), and breast cancer (MCF-7) cells compared to cisplatin (see, also, Table 1, below).

TABLE 1
Tumor Cell Growth Inhibition (IC₅₀ values, μM)

| | | Cisplatin | | Compound 1 | | Compound 2 | | Compound 3 | | Compound 4 | | Compound 5 | |
|---|---|---|---|---|---|---|---|---|---|---|---|---|---|
| | Cancer | (uM) | | (uM) | | (uM) | | (uM) | | (uM) | | (uM) | |
| Cell Line | Origin | Mean | SD | Mean | SD | Mean | SD | Mean | SD | Mean | SD | Mean | SD |
| H929 | Bone Marrow | 2.35 | 0.25 | 0.10 | 0.09 | 0.07 | | 0.47 | 0.04 | 0.05 | | 0.34 | 0.08 |
| SK-N-SH | Brain | | | 3.86 | | 2.16 | | | | 3.78 | | | |
| MCF-7 | Breast | 2.25 | 0.93 | 2.69 | 1.22 | 0.01 | 0.01 | 0.32 | 0.18 | 2.41 | 2.34 | 0.67 | 0.65 |
| MDA-MB-231 | Breast | 2.85 | 1.89 | 8.81 | 6.09 | 0.07 | 0.15 | 4.88 | 1.95 | 4.60 | 1.89 | 3.04 | 1.41 |
| Hela | Cervical | 12.32 | 5.77 | 1.39 | | 1.60 | 0.91 | 4.88 | 0.72 | 2.19 | | 1.92 | 0.36 |
| HCT-15 | Colon | 24.27 | 5.08 | 14.72 | 14.64 | 0.38 | 0.22 | 4.96 | 1.57 | 1.32 | 0.35 | 2.96 | 2.29 |
| Huh-7 | Liver | 6.47 | 1.20 | 6.20 | 5.99 | 0.81 | 0.31 | 5.66 | 1.44 | 0.75 | 0.31 | 7.81 | 4.73 |
| HepG2-PM4 | Liver | 14.04 | | 19.67 | 15.15 | 1.78 | 0.98 | 5.58 | | 3.05 | 2.05 | 6.84 | 3.69 |
| LNCap | Prostate | 10.86 | 3.72 | 2.66 | 3.41 | 0.31 | 0.18 | 1.67 | 0.56 | 0.28 | 0.22 | 1.90 | 1.10 |
| PC-3 | Prostate | 22.02 | 11.22 | 8.91 | 10.09 | 0.56 | 0.25 | 4.67 | 2.26 | 0.55 | 0.04 | 1.21 | 0.08 |

Mean and SD were calculated where multiple experiments were performed. Where mean is present without SD, only a single experiment is represented.

### Example 9. Antitumor Activity of a Combined Treatment of Cisplatin and PTBA

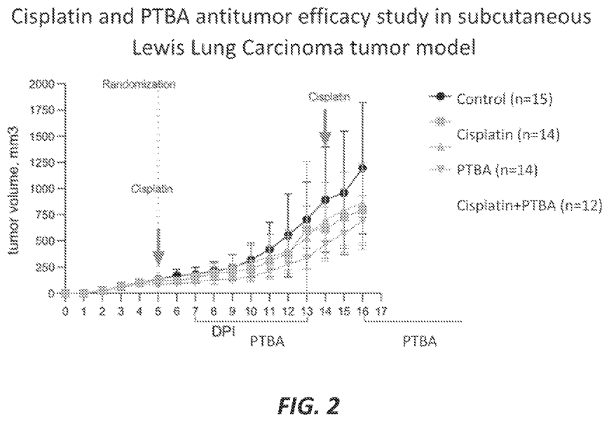

FIG. 2 illustrates the antitumor activity of an exemplary treatment of cisplatin+PTBA in Lewis Lung Carcinoma tumor model. The combination Cisplatin+PTBA treatment was more effective than either cisplatin or PTBA alone in reducing tumor size.

Example 10. Chemical conjugates of cisplatin and PTBA or PTBA analogs (i.e. HDAC8-specific inhibitors) may not only constitute a combined chemotherapeutic+epigenetic drug with dual mechanisms of action as an anti-cancer therapy, but also may also function as a combined chemotherapeutic+chemoprotective drug:

### Example 10. Toxicity Caused by Chemotherapy Drugs

Chemotherapeutic anti-cancer drugs play a vital role in the cure of patients suffering from malignancy, but are generally associated with various adverse side effects which produce significant toxicity in patients. Toxic side effects reduce the therapeutic index of the anti-cancer drug, and inadequate dosing due to intolerability of these side effects restricts the most effective use of many drugs for the treatment of malignancy.

Toxicities caused by anti-cancer drugs are generally due to ‘off-target’ effects on normal cells and tissues in the body. Organ systems commonly affected by cytotoxic anti-proliferative drugs include tissues with actively dividing cell populations, including hematopoietic and lymphoid, dermatologic, and gastrointestinal systems. Other toxic side effects of clinical importance involve renal, hepatic, pulmonary, cardiovascular, and neurologic (both central and peripheral) systems, as well as systemic reactions (infusion and anaphylactoid). In some cases, toxicity of a chemotherapy agent or immunosuppressant agent can be life-threatening, and may require supportive care that can include mechanical ventilation, vasoactive medication support, fluid resuscitation, and hemodialysis. In addition to acute toxicityacute and chronic toxicities may also occur.

Below are listed several examples of cancer chemotherapy-associated toxicity [2], including but not limited to, the following: Myelosuppression, leading to anemia, neutropenia, lymphopenia, and thrombocytopenia, is a common toxic side effect of chemotherapy, especially with use of high-dose combination regimens. Pulmonary toxicity may manifest as adult respiratory distress syndrome (ARDS), especially in patients undergoing treatment for hematologic malignancies (e.g., cytarabine or gemcitabine) and pulmonary fibrosis (eg, bleomycin). Cardiotoxic side effects of anthracyclines such as doxorubicin can lead to refractory heart failure, while treatment with 5-fluorouracil (5-FU) may precipitate symptoms of acute myocardial infarction due to vasospasm in patients with underlying risk factors for heart disease. Mucositis of the oropharynx and GI tract is painful and can lead to dehydration and malnutrition. Severe cases of oral mucositis may require intubation for airway protection. Intractable nausea, vomiting, and diarrhea can lead to dehydration, hypovolemia, and electrolyte disturbances. Nephrotoxicity is a dose-limiting side effect of cisplatin and causes renal salt wasting syndrome. During treatment with methotrexate, special attention must be paid to urinary pH to avoid precipitation in the renal tubules resulting in ATN and renal obstruction. Neurotoxicity to both peripheral and central nervous systems, including posterior reversible encephalopathy syndrome (PRES), are reported after high-dose chemotherapy. The spectrum of severity of hypersensitivity reactions ranges from flushing and rash to anaphylactic shock. Common causes of these reactions include platinum compounds, paclitaxel, and monoclonal antibodies. In addition to thrombocytopenia due to general myelotoxicity, thrombotic disorders can occur, such as venous thromboembolic disease which is a well-known complication of tamoxifen, and thrombotic thrombocytopeniaurpura (TTP) or thrombotic microangiopathy which have been associated with cisplatin, mitomycin, and gemcitabine.

Nephrotoxicity caused by chemotherapy. In particular, nephrotoxicity remains a significant complication of chemotherapeutic drugs, and can manifest in several ways, including primarily tubular-limited dysfunction, glomerular injury with proteinuria, acute kidney injury (AKI), and long-term chronic kidney disease (CKD). Furthermore, additional exposure to drugs such as aminoglycosides, nonsteroidal anti-inflammatory drugs, radiocontrast dyes, and other nephrotoxic agents, result in enhanced nephrotoxic risk when cancer chemotherapy drugs are administered concurrently.

Nephrotoxicity caused by these drugs can have multiple consequences beyond impairing renal function per se, including increased morbidity such as infectious complications, prolonged length of hospital stays, increased costs, and higher mortality rates. These acute complications are caused in part by the adverse effects of AKI itself, as well as the extra-renal toxicities of high drug levels from under-excreted drugs in the setting of reduced kidney function. Reduced drug excretion can exacerbate other toxic side effects including myelosuppression and reduced immune function, mucositis and breakdown of mucosal barriers against infection, volume depletion with hypotension from excessive vomiting and diarrhea, and other end-organ dysfunction.

Even after the onset of nephrotoxicity has been recognized, AKI often undermines the efficacy of cancer chemotherapy, due to the need to withhold, discontinue, or under-dose chemotherapeutic agents while renal function is compromised. Hemodialysis or continuous renal replacement therapy to treat nephrotoxicity can also significantly reduce circulating drug concentrations by removal from the blood, thereby further impairing the efficacy of cancer treatment.

Patients receiving cancer chemotherapy who develop severe AKI that requires dose reduction or drug discontinuation, and/or acute dialysis or continuous renal replacement therapy, all of which impedes the efficacy of chemotherapy, show increased morbidity and mortality due to progressive cancer. Mortality was reported to increase by 2-fold in critically ill patients with cancer who developed a 10% increase in serum creatinine levels, and mortality rates of hospitalized patients with a malignancy and AKI in the setting of multi-organ dysfunction range from 72%-85%, higher than those without cancer.

Patients who survive their cancer despite chemotherapy-induced nephrotoxicity are then frequently left with some level of CKD or even end-stage renal disease, requiring long-term dialysis therapy or kidney transplantation [3]. Both CKD and end-stage renal disease are associated with increased morbidity (anemia, renal osteodystrophy, cardiovascular disease, malnutrition, etc.) and mortality. Hypertension often accompanies kidney disease, increasing risk for other cardiovascular complications in addition to that associated with CKD alone. Metabolic complications associated with CKD, such as hypokalemia, hypophosphatemia, metabolic acidosis, and hypomagnesemia, may complicate therapy and cause assorted chronic conditions such as osteomalacia, osteoporosis, increased risk for cardiac arrhythmias, muscle cramping, and chronic inflammation; some of these metabolic disturbances can be permanent, depending on the agent and dose administered.

Cisplatin-induced nephrotoxicity. Of the platinum-based chemotherapeutic drugs, cisplatin (cisdiamminedichloroplatinum [II]) is the agent most frequently used, particularly for solid tumors, and has long been recognized as also one of the most nephrotoxic. Cisplatin nephrotoxicity is dose-dependent: approximately one-third of patients treated with cisplatin experience an episode of renal impairment within days after the initial dose, and renal injury worsens with repeated courses. Patients with cisplatin-associated renal injury present with non-oliguric renal failure and an inability to concentrate urine. Inability to reabsorb magnesium leads to magnesium wasting, which can be seen in over 50% of patients treated with cisplatin, and hypomagnesemia may potentiate renal cisplatin toxicity. Although AKI can recover, repeated cisplatin dosing can result in decreased urine output with progressive CKD from chronic tubulointerstitial fibrosis and chronic tubulopathies may result in irreversible renal failure.

Histologic evaluation of patients with cisplatin-associated nephrotoxicity shows that injury most frequently occurs in the cortico-medullary S3 segment of the proximal tubule, which accumulates the highest concentration of platinum, and the loop of Henle and distal tubular segments can also be affected. Cisplatin induces renal failure by promoting apoptosis of kidney tubule cells via both mitochondria-dependent and -independent pathways, in part by activation of caspase-3 in a dose-dependent manner, as well as through activation of cyclin-dependent kinases, mitogen-activated protein kinases, and p53 signaling, and in addition, cellular injury also develops from inflammation and oxidative stress.

Prevention of kidney injury has long been the primary focus of care for patients receiving cisplatin, by pre-hydration and forced diuresis with intravenous normal saline or hypertonic saline. The addition of mannitol to induce forced diuresis is sometimes used, but evidence of benefit is lacking; in some cases, this approach may cause worsened kidney function. Once cisplatin-induced nephrotoxicity develops, treatment is primarily supportive, as there are currently no effective therapies to reverse AKI or tubular dysfunction. Dialysis is reserved for advanced AKI as manifested by uremia, metabolic disturbances, and hypervolemia.

Chemoprotective agents. Treatments for drug-induced toxicities are largely supportive, but NIH National Cancer Institute (NCI) does recognize a specific class of drugs known as “chemoprotective agents”, defined as “a type of drug that helps protect healthy tissue from some of the side effects caused by certain anticancer drugs. For example, in patients receiving certain anticancer drugs, amifostine helps protect the kidneys, mesna helps protect the bladder, and dexrazoxane helps reduce heart damage”.

Accordingly, chemoprotective agents are a class of drugs that can minimize or mitigate toxicities associated with chemotherapeutic drugs without compromising their anti-cancer efficacy. To date, there are only a few examples of FDA-approved chemoprotective drugs, including amifostine, mesna, and dexrazoxane. Dexrazoxane appears to complex with metal co-factors including iron, to reduce the incidence of anthracycline-induced cardiotoxicity, allowing the delivery of higher cumulative doses of anthracyclines without incurring cardiomyopathy. Sulfur-containing nucleophiles such as amifostine and mesna, as well as glutathione, can specifically bind cisplatin- or alkylating agent-generated free radicals or alkylating agent metabolites to reduce the incidence of cisplatin-associated neurotoxicity and nephrotoxicity, or alkylating agent-associated myelosuppression and urothelial toxicity.

Mesna was approved by the FDA in 1988 for reduction of the incidence of hemorrhagic cystitis and hematuria associated with ifosfamide or cyclophosphamide for cancer chemotherapy, by detoxifying and increasing renal excretion of urotoxic metabolites of these drugs, such as acrolein. Amifostine was approved by the FDA in 1995 for reduction of the incidence of neutropenia-related fever and infection induced by DNA-binding chemotherapeutic agents including alkylating agents such as cyclophosphamide and platinum-containing agents such as cisplatin, and also used to decrease cumulative nephrotoxicity associated with platinum-containing agents.

However, the use of amifostine is limited by its adverse effects and cost [6]. Common side effects of amifostine include nausea, vomiting, diarrhea, sneezing, hiccoughs, somnolence, and hypocalcemia, while more serious side effects include hypotension (found in 62% of patients), erythema multiforme, Stevens-Johnson syndrome and toxic epidermal necrolysis, immune hypersensitivity syndrome, erythroderma, and anaphylaxis. Concerns have also been expressed about its possible interference with the therapeutic efficacy of cisplatin in some cancers, due to amifostine's cytoprotective properties.

Other approaches that have been proposed in order to mitigate platin-induced nephrotoxicity include the use of natural anti-oxidant compounds such as glutathione, selenium, Vitamin E, etc. to neutralize reactive oxygen species (ROS) that contribute to renal tubular injury, but these have not been definitively validated in large-scale clinical trials. Thus there is a need to develop newer and better agents for chemoprotection, especially against cisplatin-induced nephrotoxicity.

PTBA and its analogs as chemoprotective agents. As noted above, currently, there are no established therapies clinically proven to prevent kidney failure, promote post-injury repair, and/or reduce renal fibrosis after AKI, including cisplatin-induced AKI. Cisplatin cytotoxicity leads to an early loss of renal tubular epithelial cells due to apoptosis. Tubular epithelial cells that survive injury initially undergo transient de-differentiation followed by a period of proliferation, and re-differentiation. Subsequently, these cells repopulate damaged renal tubules with functional epithelium. Cell lineage tracing studies confirm that most of the regenerating cells after AKI are indeed derived from surviving tubular epithelial cells, and not from exogenous populations of stem cells that newly migrate into the tissue.

Recent studies have also demonstrated that a significant proportion of the surviving renal tubular epithelial cells undergo cell cycle arrest after injury, which delays tissue repair and promotes post-injury fibrosis. This inhibits repopulation of the tubules with properly redifferentiated renal epithelium, which is then replaced by non-functional fibrotic scar tissue [19]. These results indicate that cell cycle arrest plays an important role in limiting the regenerative capacity of surviving renal tubular epithelial cells, and that therapies enhancing cell cycle progression in these cells may accelerate recovery and reduce fibrosis after AKI.

In this context, HDAC inhibitors enhance survival of kidney tubule cells in the face of cisplatin-induced cellular stress by activating cAMP-responsive element binding protein (CREB)-mediated transcription, as well as by blocking the DNA damage response and associated p53 activation (which causes cell cycle arrest) during cisplatin treatment, resulting in suppression of tubular cell apoptosis. By screening a small molecule drug library, the histone deacetylase 8 (HDAC8)-specific inhibitor 4-phenylthiol-butanoic acid (PTBA) and its analogs were found to enhance proliferation of renal progenitor cells in model organisms, and to enhance recovery and reduce post-injury fibrosis after AKI by stimulating cell cycle progression in surviving renal tubular epithelial cell. In particular, PTBA and its analogs have been shown to significantly accelerate recovery of kidney function and to reduce AKI-associated morbidity and mortality, after nephrotoxin-induced injury and ischemia/reperfusion-induced kidney injury in rodents. In addition, this compound appears to reduce post-injury tubular atrophy and interstitial fibrosis after experimental AKI. Importantly, these beneficial effects on recovery of renal tubule structure and function after AKI occur when PTBA or its analog is administered 0 to 48 hours after the initial injury.

Hence, these preclinical studies provided evidence that PTBA and its analogs act as repair-promoting regenerative agents that are effective even when administered days after acute renal insult. Accordingly, by combining PTBA or its analogs with cisplatin through chemical conjugation, not only will the conjugate drug have the potential to act as a synergistic anti-cancer drug combination in its own right, but PTBA and its analogs will also provide for chemoprotection against nephrotoxicity during cancer therapy. Additionally, the chemoprotective effects of PTBA and its analogs, acting via specific inhibition of HDAC8, may extend beyond preventing or mitigating nephrotoxicity. Additional chemoprotective effects against various other toxic side effects of cisplatin and other chemotherapy drugs, including but not limited to myelotoxicity, mucositis, and neurotoxicity, are suggested (e.g. through suppression of inflammatory cytokine production as has been reported for another HDAC8-specific inhibitor compound. Preliminary results also suggest a chemoprotective effect of PTBA against cisplatin-induced myelotoxicity). By reducing adverse side effects, this chemoprotective characteristic of PTBA and its analogs, co-administered as a chemical conjugate with cisplatin, has the potential to increase drug tolerability, to permit uninterrupted treatment schedules, and to eliminate the need to reduce drug doses. Hence this invention has considerable potential to improve treatment efficacy and improve the quality of life for cancer patients.

Derivatives of 4-(phenylthio)butanoic acid (PTBA), 4-(phenylthio)butyryl. Some embodiments of the present disclosure include one or more derivatives of 4-(phenylthio)butanoic acid or 4-(phenylthio)butyrate (PTBA), including 4-(phenylthio)butyryl

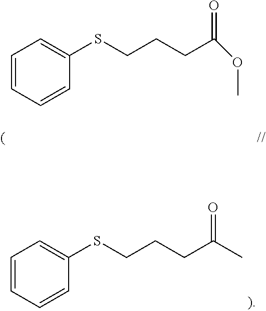

In some embodiments, the derivative of PTBA can be or comprise 4-(phenylthio)butyryl. Some embodiments of the present disclosure include one or more derivatives of PTBA or 4-(phenylthio)butyryl In some embodiments, the derivative of PTBA or derivative of 4-(phenylthio)butyryl) can be or comprise 1-5 phenyl ring substituents on the terminal phenyl ring of PTBA or 4-(phenylthio)butyryl. Such substituents at the phenyl ring can be or comprise, for example, halo (Cl, F, Br, etc.), alkyl (methyl, ethyl, propyl, isopropyl, etc.), alkoxy (e.g., methoxy), hydroxy (e.g., OH), hydroxyalkyl (e.g. hydroxymethyl (CH₂OH)), CF3, CN. Derivatives of PTBA can also or alternatively comprise one or more substitutions in the backbone (e.g., at a backbone methylene). Such backbone substituents can be or comprise, for example, alkyl (methyl, ethyl, propyl, isopropyl, etc.), alkoxyl (e.g., methoxy, ethoxy, etc.), or halo (e.g., fluoro, chloro, bromo, etc.).

In some embodiments, the terminal phenyl group of the PTBA or 4-(phenylthio)butyryl may be or comprise an alternative ring system, such as an alternative aromatic ring system as described herein. Illustratively, the alternative ring system can be selected from the group consisting of an optionally substituted cycloalkyl, cycloalkene, cyclodiene, heterocyclic alkyl, heterocyclic alkene, heterocyclic diene, or optionally substituted aromatic ring system, as described herein (e.g., phenyl, phenoxy, aryl, heterocyclic aryl, pyridyl, pyrimidinyl, pyrazinyl, naphthyl, benzyl, benzyloxy, benzodiazol, benzothiazole, methoxyphenyl, methylthiophenyl, α,α-dimethylbenzyl, 1H-1,3-benzodiazol-2-yl, 1,3-benzothiazol-2-yl, or 1-methyl-1H-1,3benzodiazol-2-yl). In some embodiments, the terminal phenyl group or alternative (aromatic) ring system may be optionally substituted at one or more positions.

Illustratively, up to 5 substituents can optionally be present on the terminal phenyl group or alternative (aromatic) ring system. Each substituent can be optionally independently selected from an “R-group” according to the present disclosure. In some embodiments, adjacent substituents present on the terminal phenyl group or alternative (aromatic) ring system together form an additional, optionally substituted ring or aromatic ring (or ring system). Illustrative, adjacent substituents present on the terminal phenyl group or alternative (aromatic) ring system together may form an optionally substituted cycloalkyl, cycloalkene, cyclodiene, heterocyclic alkyl, heterocyclic alkene, heterocyclic diene, or optionally substituted aromatic ring system, as described herein (e.g., phenyl, phenoxy, aryl, heterocyclic aryl, pyridyl, pyrimidinyl, pyrazinyl, naphthyl, benzyl, benzyloxy, benzodiazol, benzothiazole, methoxyphenyl, methylthiophenyl, α,α-dimethylbenzyl, 1H-1,3-benzodiazol-2-yl, 1,3-benzothiazol-2-yl, or 1-methyl-1H-1,3benzodiazol-2-yl).

Additional/Alternative Ligands (“L”). In some embodiments, the (additional) ligand (“L”) in Formulas A-G, or other aspect or embodiment of the present disclosure, can be or comprise OR₂. In some embodiments, ligands (“L”) can or comprise be NR₂ or NHR₂. Illustratively, R₂ can be selected from any suitable “R-group” as known in the art and/or disclosed herein. Those skilled in the art will appreciate that other R-groups may be suitable as substituents for the disclosed R-groups.

Illustratively, a ligand “(L”), or R₂, according to the present disclosure may be selected from the group consisting of: nothing; hydrogen; OH; halo; alkyl (e.g., methyl; C1-6 alkyl); CH(CH3)2; CH2C6H5; CH2CH(CH3)2; CH(CH3)CH2CH3; CHO; CH2OH; CONH2; OCH2COOH; CH3CH(OH); (CH2)2SO3H; NO2; CN; optionally substituted alkyl, cycloalkyl, cycloalkene, cyclodiene, heterocyclic alkyl, heterocyclic alkene, heterocyclic diene, keto, alkoxy (e.g., methoxy, C1-6 alkoxy), thiol, thioalkyl, sulfone, sulfoxide, sulfoxyalkyl, sulfonylalkyl, alkylene dioxy, acetyl, acetoxy, haloalkyl, haloalkoxy, acetoxy, N(alkyl)2, aromatic ring system (e.g., phenyl, phenoxy, aryl, heterocyclic aryl, pyridyl, pyrimidinyl, pyrazinyl, naphthyl, benzyl, benzyloxy, benzodiazol, benzothiazole, methoxyphenyl, methylthiophenyl, α,α-dimethylbenzyl, 1H-1,3-benzodiazol-2-yl, 1,3-benzothiazol-2-yl, or 1-methyl-1H-1,3benzodiazol-2-yl); CHZCOOH, wherein Z is selected from the group consisting of an “R-group” according to the present disclosure; and combinations thereof. Illustrative cyclic or aromatic ring system R-groups may have one more substituents (e.g., up to 5 substituents) optionally present on the ring or aromatic ring system and optionally independently selected from an “R-group” according to the present disclosure. In some embodiments, adjacent substituents present on the ring or aromatic ring system together form an additional, optionally substituted ring or aromatic ring or ring system. Illustrative, adjacent substituents present on the additional, optionally substituted ring or aromatic ring or ring system together may form an optionally substituted cycloalkyl, cycloalkene, cyclodiene, heterocyclic alkyl, heterocyclic alkene, heterocyclic diene, or optionally substituted aromatic ring system, as described herein (e.g., phenyl, phenoxy, aryl, heterocyclic aryl, pyridyl, pyrimidinyl, pyrazinyl, naphthyl, benzyl, benzyloxy, benzodiazol, benzothiazole, methoxyphenyl, methylthiophenyl, α,α-dimethylbenzyl, 1H-1,3-benzodiazol-2-yl, 1,3-benzothiazol-2-yl, or 1-methyl-1H-1,3benzodiazol-2-yl).

In some embodiments, in compounds of the invention, at least two of the ligands (“L”) are identical.

In some embodiments, the (additional) ligand (“L”) in Formulas A-G, or other aspect or embodiment of the present disclosure, can be selected from ligands designated herein L1 through L47, below:

L1,

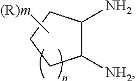

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; wherein n being an integer between 0 and 2, wherein m being an integer between 0 and 5; and wherein the amine groups may be trans or cis to each other; and wherein the chiral carbons to which the amine groups are bonded may be R,R; R,S; or S,S; and wherein the ligand associates to the Pt via the amine moieties;

L2,

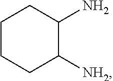

wherein the amine groups may be trans or cis to each other; and wherein the chiral carbons to which the amine groups are bonded may be R,R; R,S; or S,S; and wherein the ligand associates to the Pt via the amine moieties;

L3,

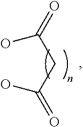

wherein n being an integer between 0 and 5; wherein the ligand associates to the Pt via the oxygen atoms;

L4,

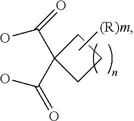

wherein R is selected from selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and n being an integer between 0 and 2, and wherein m being an integer between 0 and 5; wherein the ligand associates to the Pt via the oxygen atoms;

L5, —NH₂—R, wherein R is null or selected from hydrogen, substituted or unsubstituted alkyl, substituted or unsubstituted cycloalkenyl, substituted or unsubstituted cycloalkynyl, substituted or unsubstituted cycloalkyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; wherein the ligand associates to the Pt via the amine moiety;

L6,

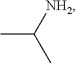

wherein the ligand associates to the Pt via the amine moiety;

L7,

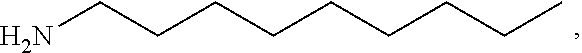

wherein the ligand associates to the Pt via the amine moiety;

L8,

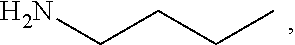

wherein the ligand associates to the Pt via the amine moiety;

L9,

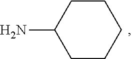

wherein the ligand associates to the Pt via the amine moiety;

L10, —NHRR′, wherein R and R′ are each independently selected from hydrogen, substituted or unsubstituted alkyl, substituted or unsubstituted cycloalkenyl, substituted or unsubstituted —NHRR′ cycloalkynyl, substituted or unsubstituted cycloalkyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R₂, substituted or unsubstituted —OR₃, substituted or unsubstituted —SR₄, substituted or unsubstituted —S(0)Rs, substituted or unsubstituted alkylene-COOH, substituted or unsubstituted ester, OH, —SH, and —NH, phenyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; wherein the ligand associates to the Pt via the amine moiety;

L11,

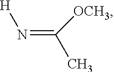

wherein the ligand associates to the Pt via the amine moiety;

L12, —NC(0)OR, wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl, and wherein n being an integer between 0 and 5; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; wherein the ligand associates to the Pt via the amine moieties;

L13,

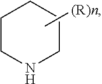

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

L14,

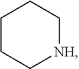

wherein the ligand associates to the Pt via the amine moiety;

L15,

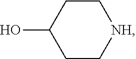

wherein the ligand associates to the Pt via the amine moiety;

L16,

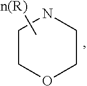

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

L17,

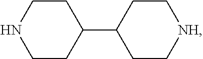 [SMILES: C1CC(C2CCNCC2)CCN1]

wherein the ligand associates to the Pt via the amine moiety;

L18,

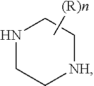

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

L19,

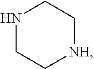

wherein the ligand associates to the Pt via the amine moiety;

L20,

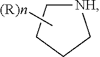

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

L21,

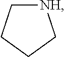

wherein the ligand associates to the Pt via the amine moiety;

L22,

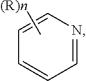

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

L23,

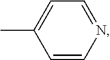

wherein the ligand associates to the Pt via the amine moiety;

L24,

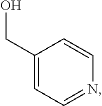

wherein the ligand associates to the Pt via the amine moiety;

L25,

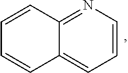

wherein the ligand associates to the Pt via the amine moiety;

L26,

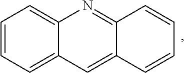 [SMILES: c1ccc2nc3ccccc3cc2c1]

wherein the ligand associates to the Pt via the amine moiety;

L27,

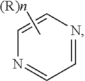

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

L28,

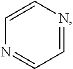

wherein the ligand associates to the Pt via the amine moiety;

L29,

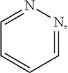

wherein the ligand associates to the Pt via the amine moiety;

L30,

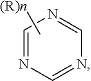

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

L31,

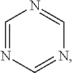

wherein the ligand associates to the Pt via the amine moiety;

L32,

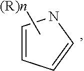

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

L33,

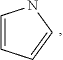

wherein the ligand associates to the Pt via the amine moiety;\

L34,

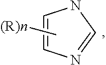

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

L35,

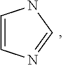

wherein the ligand associates to the Pt via the amine moiety;

L36,

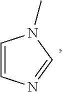

wherein the ligand associates to the Pt via the amine moiety;

L37,

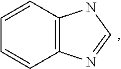

wherein the ligand associates to the Pt via the amine moiety;

L38,

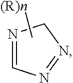

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

L39,

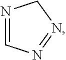

wherein the ligand associates to the Pt via the amine moiety;

L40,

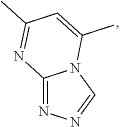

wherein the ligand associates to the Pt via the amine moiety;

L41,

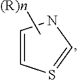

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine moieties;

L42,

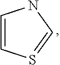

wherein the ligand associates to the Pt via the amine moiety;

L43,

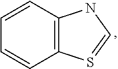

wherein the ligand associates to the Pt via the amine moiety;

L44, -S(O)RR', wherein R and R′ are each independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR1R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the sulfinyl moieties (S atom);

L45,

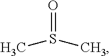

wherein the ligand associates to the Pt via the sulfinyl moiety (S atom);

L46,

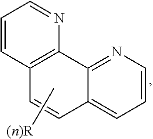

wherein R is selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl, halogen, substituted or unsubstituted —NR₁R2, substituted or unsubstituted —OR3, substituted or unsubstituted —SR4, substituted or unsubstituted —S(0)Rs, substituted or unsubstituted alkylene-COOH, substituted or unsubstituted ester, OH, —SH, and —NH, phenyl, hydroxyl; wherein each of Ri, R2, R3, R4 and R5 is independently selected from hydrogen, substituted or unsubstituted alkyl, substituted or unsubstituted cycloalkyl, substituted or unsubstituted cycloalkenyl, substituted or unsubstituted cycloalkynyl, substituted or unsubstituted heterocyclyl, substituted or unsubstituted aryl, substituted or unsubstituted heteroaryl, substituted or unsubstituted heterocyclyl, substituted or unsubstituted alkenyl, substituted or unsubstituted alkynyl; and wherein n being an integer between 0 and 5; wherein the ligand associates to the Pt via the amine atoms;

L47,

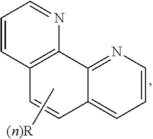

wherein the ligand associates to the Pt via the amine atoms.

In each of the above ligands, the various substituting groups or radicals are selected as defined herein.

In some embodiments, at least one of the ligands is halide (Cl, Br, I or F). In some embodiments, at least one of the ligands is Cl.

In some embodiments, at least one of the ligands is an amine selected from ammonia, a primary amine, a secondary amine, a non-planar heterocyclic aliphatic amine or a heterocyclic aromatic amine. In some embodiments, at least one of the ligands is —NH3. In some embodiments, at least one of the ligands is —NH2.

In some embodiments, at least one of the ligands is a primary amine. Non-limiting examples of a primary amine are methylamine, ethylamine, n-propylamine, isopropylamine, n-butylamine, n-hexylamine, n-heptylamine or n-nonylamine.

In some embodiments, at least one of the ligands is a secondary amine. Non limiting examples of a secondary amine are dimethylamine, diethylamine, dipropylamine, dibutylamine.

In some embodiments, at least one of the ligands is a non-planar heterocyclic aliphatic amine. Non-limiting examples of a non-planar heterocyclic aliphatic amine are piperazine, 2-methylpiperazine, piperadine, 2-, 3- or 4-hydroxypiperidine, 4-piperidino-piperidine, pyrrolidine, 4-(2-hydroxyethyl)piperazine or 3-aminopyrrolidine.

In some embodiments, at least one of the ligands is a heterocyclic aromatic amine. Non-limiting examples of a heterocyclic aromatic amine are pyridine, 2-, 3-, or 4-aminopyridine, 2-, 3-, or 4-picoline, quinoline, 3-, or 4-aminoquinoline, thiazole, imidazole, 3-pyrroline, pyrazine, 2-methylpyrazine, 4-aminooquinaldine, pyridazine, 1,10-phenanthroline and 5,6-dimethyl-1,10-phenanthroline.

### Conclusion

While the foregoing detailed description makes reference to specific exemplary embodiments, the present disclosure may be embodied in other specific forms without departing from its spirit or essential characteristics. Accordingly, the described embodiments are to be considered in all respects only as illustrative and not restrictive. For instance, various substitutions, alterations, and/or modifications of the inventive features described and/or illustrated herein, and additional applications of the principles described and/or illustrated herein, which would occur to one skilled in the relevant art and having possession of this disclosure, can be made to the described and/or illustrated embodiments without departing from the spirit and scope of the disclosure as defined by the appended claims. Such substitutions, alterations, and/or modifications are to be considered within the scope of this disclosure.

The scope of the invention is, therefore, indicated by the appended claims rather than by the foregoing description. The limitations recited in the claims are to be interpreted broadly based on the language employed in the claims and not limited to specific examples described in the foregoing detailed description, which examples are to be construed as non-exclusive and non-exhaustive. All changes which come within the meaning and range of equivalency of the claims are to be embraced within their scope.

It will also be appreciated that various features of certain embodiments can be compatible with, combined with, included in, and/or incorporated into other embodiments of the present disclosure. For instance, systems, methods, and/or products according to certain embodiments of the present disclosure may include, incorporate, or otherwise comprise features described in other embodiments disclosed and/or described herein. Thus, disclosure of certain features relative to a specific embodiment of the present disclosure should not be construed as limiting application or inclusion of said features to the specific embodiment.

In addition, unless a feature is described as being requiring in a particular embodiment, features described in the various embodiments can be optional and may not be included in other embodiments of the present disclosure. Moreover, unless a feature is described as requiring another feature in combination therewith, any feature herein may be combined with any other feature of a same or different embodiment disclosed herein. It will be appreciated that while features may be optional in certain embodiments, when features are included in such embodiments, they can be required to have a specific configuration as described in the present disclosure.

Likewise, any steps recited in any method or process described herein and/or recited in the claims can be executed in any suitable order and are not necessarily limited to the order described and/or recited, unless otherwise stated (explicitly or implicitly). Such steps can, however, also be required to be performed in a specific order or any suitable order in certain embodiments of the present disclosure.

Furthermore, various well-known aspects of illustrative systems, methods, products, and the like are not described herein in particular detail in order to avoid obscuring aspects of the example embodiments. Such aspects are, however, also contemplated herein.

We claim:

1. A compound according to Formula I: 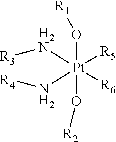
wherein:
R₁ is 4-(phenylthio)butyryl 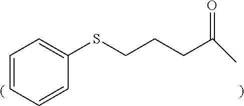
or a derivative of 4-(phenylthio)butyryl;
R₂ is selected from the group consisting of 4-(phenylthio)butyryl, H, COCH₃ 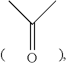
CO(CH₂)₂CH₃, 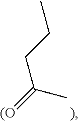
and (COCH((CH₂)₂CH₃)₂ 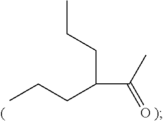
R₃ is H or, together with R₄, forms 1,2-cyclohexyl 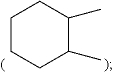
R₄ is H or, together with R₃, forms 1,2-cyclohexyl 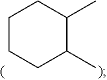
R₅ is Cl or, together with R₆, forms oxalate 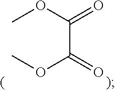
and
R₆ is Cl or, together with R₅, forms oxalate

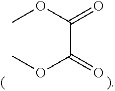

2. The compound of claim 1, wherein R₃ is H, R₄ is H, R₅ is Cl, and R₆ is Cl.

3. The compound of claim 2, wherein R₂ is 4-(phenylthio)butyryl or H.

4. The compound of claim 2, wherein R₂ is 4-(phenylthio)butyryl.

5. The compound of claim 2, wherein R₂ is H.

6. The compound of claim 2, wherein R₂ is selected from the group consisting of COCH₃ 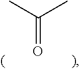
CO(CH₂)₂CH₃, 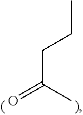
and (COCH((CH₂)₂CH₃)₂

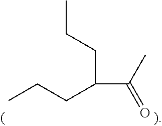

7. The compound of claim 2, wherein R₂ is COCH₃

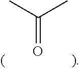

8. The compound of claim 2, wherein R₂ is CO(CH₂)₂CH₃,

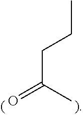

9. The compound of claim 2, wherein R₂ is (COCH((CH₂)₂CH₃)₂

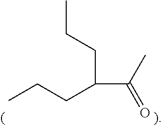

10. The compound of claim 1, wherein R₂ is 4-(phenylthio)butyryl or H.

11. The compound of claim 1, wherein R₂ is 4-(phenylthio)butyryl.

12. The compound of claim 1, wherein R₂ is H.

13. The compound of claim 1, wherein R₂ is selected from the group consisting of COCH₃ 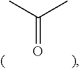
CO(CH₂)₂CH₃, 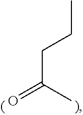
and (COCH((CH₂)₂CH₃)₂

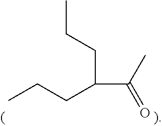

14. The compound of claim 1, wherein R₂ is COCH₃

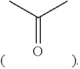

15. The compound of claim 1, wherein R₂ is CO(CH₂)₂CH₃,

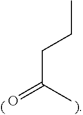

16. The compound of claim 1, wherein R₂ is (COCH((CH₂)₂CH₃)₂

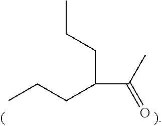

17. The compound of claim 1, wherein R₃ is H and R₄ is H.

18. The compound of claim 1, wherein R₅ is Cl and R₆ is Cl.

19. The compound of claim 1, wherein:
R₃, together with R₄, forms 1,2-cyclohexyl 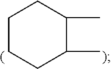
R₄, together with R₃, forms 1,2-cyclohexyl 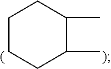
R₅, together with R₆, forms oxalate 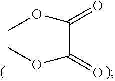
and
R₆, together with R₅, forms oxalate

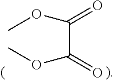

20. The compound of claim 19, wherein R₂ is H or COCH₃

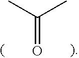

21. The compound of claim 19, wherein R₂ is H.

22. The compound of claim 19, wherein R₂ is COCH₃

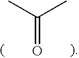

23. The compound of claim 1, wherein:
R₃, together with R₄, forms 1,2-cyclohexyl 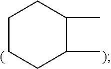
and
R₄, together with R₃, forms 1,2-cyclohexyl

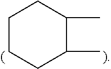

24. The compound of claim 23, wherein R₂ is H.

25. The compound of claim 23, wherein R₂ is COCH₃

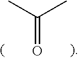

26. The compound of claim 1, wherein:
R₅, together with R₆, forms oxalate 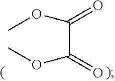
and
R₆, together with R₅, forms oxalate

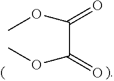

27. The compound of claim 26, wherein R₂ is H.

28. The compound of claim 26, wherein R₂ is COCH₃

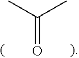

29. The compound of any one of claims 1-28, wherein R₁ is 4-(phenylthio)butyryl.

30. The compound of any one of claims 1-28, wherein R₁ is a derivative of 4-(phenylthio)butyryl, wherein the derivative comprises 4-(phenylthio)butyryl having one or more phenyl ring substituents, optionally selected from the group consisting of halo (Cl, F, Br, etc.), alkyl (methyl, ethyl, propyl, isopropyl, etc.), alkoxy (e.g., methoxy), hydroxy (e.g., OH), hydroxyalkyl (e.g. hydroxymethyl (CH₂OH)), CF₃, CN.

31. The compound of any one of claims 1-28, wherein R₁ is a derivative of 4-(phenylthio)butyryl, wherein the derivative comprises 4-(phenylthio)butyryl having one or more backbone substituents or backbone methylene substitutes, optionally selected from the group consisting of alkyl, alkoxyl, and halo.

32. The compound of claim 1, wherein the compound is selected from the group consisting of:

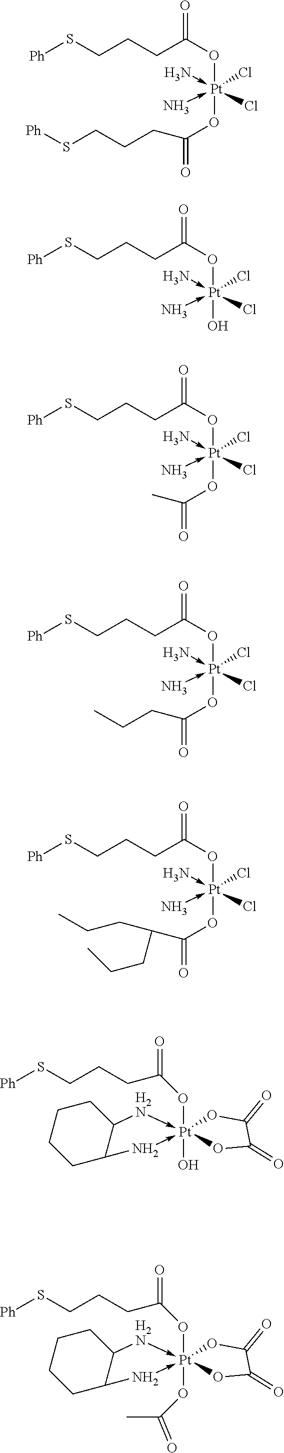

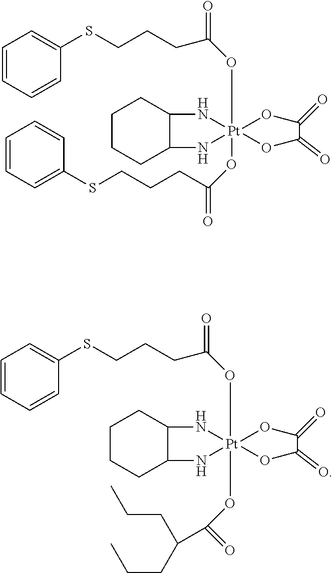

33. A compound, comprising:
a platinum-based chemotherapeutic molecule, preferably selected from the group consisting of cisplatin and oxaliplatin; and
one or more molecule or moiety of 4-(phenylthio)butanoic acid (PTBA) conjugated to the platinum-based chemotherapeutic molecule; and
optionally, one or more additional ligand conjugated to the platinum-based chemotherapeutic molecule.

34. A compound, comprising:
a platinum-based chemotherapeutic molecule, preferably selected from the group consisting of cisplatin and oxaliplatin; and
a molecule or moiety of 4-(phenylthio)butanoic acid (PTBA) conjugated to the platinum-based chemotherapeutic molecule; and
a ligand conjugated to the platinum-based chemotherapeutic molecule.

35. The compound of claim 34, wherein the ligand is selected from the group consisting of 4-(phenylthio)butyryl, OH, OCOCH₃ 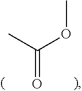
OCO(CH₂)₂CH₃, 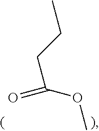
and O(COCH((CH₂)₂CH₃)₂

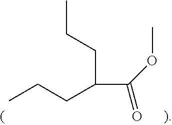

36. A composition comprising:
the compound of any one of claims 1-35; and
a carrier or excipient.

37. The composition of claim 36 for use as a medicament for the treatment of cancer or a tumor.

38. The composition of claim 36 for use as a medicament for the treatment of cancer or a tumor, wherein the cancer or tumor is suitable for or susceptible to treatment with cisplatin or oxaliplatin.

39. The composition of claim 36 for use in the treatment of cancer or a tumor.

40. The composition of claim 36 for use in the treatment of cancer or a tumor, wherein the cancer or tumor is suitable for or susceptible to treatment with cisplatin or oxaliplatin.

41. A method, comprising administering the composition of claim 36 to a subject.

42. A method for treating cancer or a tumor, the method comprising administering the composition of claim 36 to a subject having the cancer or tumor.

43. The method of claim 42, wherein the cancer or tumor is suitable for or susceptible to treatment with cisplatin or oxaliplatin.

44. A method of inhibiting histone deacetylase (HDAC) or HDAC activity, the method comprising administering the composition of claim 36 to a subject.

45. A method of reducing tumor size, the method comprising administering the composition of claim 36 to a subject having a tumor.

46. The method of claim 45, wherein the tumor is suitable for or susceptible to treatment with cisplatin or oxaliplatin.

47. A method, comprising administering the compound of any one of claims 1-35 to a subject.

48. A method for treating cancer or a tumor, the method comprising administering the compound of any one of claims 1-35 to a subject having the cancer or tumor.

49. The method of claim 48, wherein the cancer or tumor is suitable for or susceptible to treatment with cisplatin or oxaliplatin.

50. A method of inhibiting histone deacetylase (HDAC) or HDAC activity, the method comprising administering the compound of any one of claims 1-35 to a subject.

51. A method of reducing tumor size, the method comprising administering the compound of any one of claims 1-35 to a subject having a tumor.

52. The method of claim 51, wherein the tumor is suitable for or susceptible to treatment with cisplatin or oxaliplatin.

53. The compound of any one of claims 1-35 for use in the manufacture of a medicament for the treatment of cancer or tumor.

54. The compound of any one of claims 1-35 for use in the manufacture of a medicament for the treatment of cancer or a tumor, wherein the cancer or tumor is suitable for or susceptible to treatment with cisplatin or oxaliplatin.

55. The compound of any one of claims 1-35 for use in the treatment of cancer or a tumor.

56. The compound of any one of claims 1-35 for use in the treatment of cancer or a tumor, wherein the cancer or tumor is suitable for or susceptible to treatment with cisplatin or oxaliplatin.
